(19) United States
(12) Patent Application Publication
(10) Pub. No.: US 2023/0078644 A1
(43) Pub. Date: Mar. 16, 2023

(54) DEVICE AND METHOD FOR DETECTING NUCLEIC ACIDS IN BIOLOGICAL SAMPLES

(71) Applicant: Definitive Biotechnologies LLC, Elkridge, MD (US)

(72) Inventors: Timothy Alcorn, Raleigh, NC (US); Robert Altavela, Webster, NY (US); Michael Carlotta, Lilburn, GA (US); David Cigna, Rochester, NY (US); John C. Detter, Melbourne Beach, FL (US); Steven Dietl, Ontario, NY (US); Charles Facchini, Webster, NY (US); Todd Haran, Bloomfield, NY (US); Roger Markham, Webster, NY (US); Michael Murray, Bloomfield, NY (US); Scott Rosebrough, Avon, NY (US); Jeffrey Serbicki, Holley, NY (US); Qing Yang, Pittsford, NY (US)

(21) Appl. No.: 17/941,816

(22) Filed: Sep. 9, 2022

Related U.S. Application Data
(60) Provisional application No. 63/243,005, filed on Sep. 10, 2021.

(51) Int. Cl. B01L 3/00; C12Q 1/6806; B01L 7/00

(52) U.S. Cl. CPC B01L 3/502753; C12Q 1/6806; B01L 3/5029; B01L 7/52; B01L 3/502723; B01L 3/502715; B01L 3/502738; B01L 2200/04; B01L 2400/0487; B01L 2400/0694; B01L 2300/06; B01L 2200/0652; B01L 2200/0684; B01L 2300/165; B01L 2300/168; B01L 2200/0689; B01L 2200/10; B01L 2200/085; B01L 2300/044

(57) ABSTRACT

A device for detecting nucleic acids in a biological sample has a sample port, a lysis station and a sample conduit configured to mix a sample and lysis agent to form a sample-lysis mixture, pass the sample-lysis mixture across a solid-state membrane to capture nucleic acids in the biological sample therein, and receive the remainder of the sample-lysis mixture in a waste chamber. The wash station is configured to introduce the wash solution following the sample-lysis mixture, pass the wash solution across the solid-state membrane to purify nucleic acids captured therein, and receive the wash solution from the solid-state membrane in the waste chamber. The elution station is configured to pass the eluent across the solid-state membrane, elute captured nucleic acids from the solid-state membrane, and pass the captured nucleic acids into one or more reaction chambers for amplifying and detecting the captured nucleic acids.

### CROSS-REFERENCE TO RELATED PATENT APPLICATIONS

This patent application claims priority under 35 U.S.C. § 119(e) to U.S. provisional patent application Ser. No. 63/243,005, filed Sep. 10, 2021, entitled “Device and Method for Detecting Nucleic Acids in Biological Samples,” which is hereby incorporated by reference in its entirety as part of the present disclosure. This patent application also includes subject matter related to co-pending U.S. patent application Ser. No. 17/647,828, filed Jan. 12, 2022, entitled “Device and Method for Detecting Nucleic Acids in Biological Samples,” which is assigned to the assignee of the present invention and is hereby incorporated by reference in its entirety as part of the present disclosure.

### GOVERNMENT LICENSE RIGHTS

This invention was made with government support under FA864921P09 awarded by the U.S. Air Force. The government has certain rights in the invention.

### FIELD OF THE INVENTION

The present invention is directed to devices for and methods of isolating, concentrating, amplifying, and detecting nucleic acids in biological samples such as saliva, blood, or urine samples and, more particularly, to such devices or methods including solid-state membranes and microfluidic reaction chambers and methods of collecting and processing such biological samples.

### BACKGROUND INFORMATION

A prior art isothermal nucleic acid amplification reactor with an integrated solid-state membrane is shown in U.S. Pat. No. 9,796,176 (“the '176 patent”). The '176 patent discloses a microfluidic cassette that integrates nucleic acid capture, concentration, purification, isothermal amplification, and real-time fluorescence detection into a single reaction chamber. As shown in FIG. 11 of the '176 patent, a Flanders Technologies Associates or FTATM membrane plug is mounted at the base of the reaction chamber, and pouches 1, 2 and 3 are connected to the reaction chamber above the FTA membrane. The total volume of the reaction chamber is about 20 μl. Sample material is added to the reaction chamber. Lysis buffer in pouch 1 is added to the reaction chamber by compressing pouch 1. The lysate mixture is incubated for a prescribed time, with optional stirring by magnetic rods that may be turned by a rotating magnet. Next, an absorbent sink pad is contacted to the FTA membrane, which wicks in lysed sample to the absorption sink pad. Nucleic acid is adsorbed on the FTA membrane plug. Next, pouch 2 is compressed to add wash buffer to the reaction chamber. Then, the absorbent pad is again contacted to the FTA membrane to wick the wash buffer through the membrane. Next pouch 3 is compressed to fill the chamber with molecular or de-ionized water. The chamber is then heated by an external heating element or by chemical heating (exothermic reaction). The heating releases pre-stored, encapsulated reagents for isothermal nucleic acid amplification. This can be achieved by encapsulating the dry reagents with low melting point paraffin, which melts upon heating the reaction chamber to the desired incubation temperature (e.g., 60° C.) and releases the reagents for amplification. The amplification step proceeds at elevated temperatures for about 20-60 minutes. After amplification, a lateral flow strip is contacted to a porous membrane plug of the reaction chamber. This is made of a material that has low nucleic acid binding. The strip is loaded with amplification product, which is functionalized with antibody or antigen to capture the labeled amplicon. The LF strip loading pad contains reporter particles to enhance detection of product captured on the strip.

One drawback associated with the above-described prior art is that the solid-state membrane is fixedly mounted within a fixed fluid conduit to the reaction chamber, and thus the biological sample, lysate mixture and wash buffers are first introduced into the reaction chamber, and then wicked through the membrane and absorbed by the absorbent sink pad. As a result, the volumes of the biological sample, lysate mixture and wash buffers are limited by the capacity of the reaction chamber and absorbent sink pad. As indicated above, the total volume of the reaction chamber is about 20 μl. This limits the ability to add higher sample volumes in order to increase the ability to detect targets that may be in small or low concentrations in the original sample. The small volume limits the amount of sample which can be tested and decreases the ability of the test to detect dilute or low concentration nucleic acid targets. Yet another potential drawback is that the reaction chamber may contain dry reagents encapsulated in a low-melting point paraffin for release during the heating and nucleic acid amplification step. Because the lysed sample and the wash buffers must all flow through the reaction chamber, the lyophilized reagents must be sealed in paraffin to prevent premature hydrolyzation and the loss of reagent before the reaction. The paraffin may upset the purity of the reagents and reduce the sensitivity of the assay. In addition, the encapsulated reagents contained within the reaction chamber may further limit the available volume of the reaction chamber for the above-described fluids required for the preceding steps. As a result, smaller volumes of biological samples may be passed across the membrane, and lesser amounts of target nucleic acids may be captured, purified and amplified, than desired. Yet another drawback is that the capture, purification and amplification of lesser amounts of targeted nucleic acids than desired, may lead to less sensitive detection of, and testing for, such targeted nucleic acids. In other words, it would be desirable for a device or method to allow for greater volumes of biological samples to be passed across such a membrane, to in turn allow for the capture of greater amounts of targeted nucleic acids to thereby improve the ability to detect such nucleic acids. It would also be desirable to have a system that does not require paraffin or like sealant to prevent hydrolyzation.

Some in vitro diagnostic molecular biology devices contain blisters filled with liquid reagents. These blisters are usually formed with a semi-stiff cold formed top dome and a more flexible lidding film that is added and heat sealed after filling. The blister must be compressed with an actuator that deforms the top film and in turn expands the flexible lidding film so that is comes into contact with a rupture pin. The force required to perform this action can be greater than desired, especially in the case of a human or manually-actuated blister. The top blister is then continued to be compressed and further deformed until all or most of the liquid is expelled into the microfluidic device.

As indicated above, a solid-state, glass fiber membrane is commonly used to capture RNA from a sample solution. In that process ethanol is used to promote RNA retention to the membrane and water is used to release the RNA from the membrane and deliver the captured RNA to a LAMP reaction chamber where amplification occurs in a process known as elution. For amplification to occur, it is critical that substantially no ethanol is delivered to the amplification chamber. It is challenging to do that because some ethanol is retained in the solid-state membrane and released into the water during elution. This is referred to as ethanol poisoning. One way to eliminate ethanol poisoning is to first dry the ethanol from the membrane before elution. However, in a lab on chip system, it can be difficult or challenging to dry the membrane before elution.

There are two primary means of making visual detection of RNA amplification. One is color change which means that the sample's spectral absorption changes, and accordingly, the color of light reflected by the sample also changes. In a simplified example, if a sample which reflects green light changes to absorb more green light, the sample will appear less green to an observer. When the change in absorption is within the visual spectrum, a human observer may be able to detect the change. Sometimes an illumination light is used such that the incoming light is filtered by the medium and then directed to the observer. Another means of visual detection is by fluorescence. In this case, the sample is exposed to light that stimulates fluorescence and the observer selectively observes the fluorescence. When the stimulating light is ultraviolet, human eyes can perform the observation, as the ultraviolet light will not interfere. However, if the stimulating light and the fluorescent light have sufficiently similar wavelengths, human eyes are not able to detect the presence of the fluorescent light with accuracy, and a tool is required to aid the human filter out the stimulating light. One means of suppressing the observation of the stimulating light is by the use of sharp cutoff or passband filters, which selectively allow only certain wavelengths of light to pass through. A passband filter could thus allow the fluorescent light to pass through while blocking the stimulating light, even if the wavelengths are too close for the human eye to discern the difference. However, passband filters can be expensive, particularly when the two wavelengths are closely matched.

There are several known methods for the collection of human saliva. One method is referred to as a drool method where the patient spits directly into a cup or collection vessel. After the saliva is collected, a transfer device, such as a pipette, is required to meter out saliva from the collection vessel. Then, another mechanism is required to push the transferred sample into a lysis reservoir, and yet another mechanism may be required to seal the lysis reservoir after the sample is transferred therein. Another method uses an absorbent pad to collect the saliva. The saliva sample is transferred from the pad to a preservation buffer by allowing the absorbent pad to soak in the preservation buffer for several minutes. After the saliva is transferred to the preservation buffer, the same sequence of steps as mentioned above may be required to transfer the sample into a lysis reservoir and to seal the reservoir.

The foregoing steps relating to the preparation of saliva samples are a potential source of testing error, and require a trained operator or relatively complex processes to mitigate risks related to cross-contamination among samples and to ensure containment of harmful pathogens that may be contained within the samples. In addition, such processes of preparing biological samples involve interaction with hazardous chemical mixtures, including lysing agents which break down cell membranes. Due to these limitations, prior art devices are generally not suitable for use by the general public, limiting their utility and the market for such devices.

It is an object of the present invention, and/or of the currently preferred embodiments thereof, to overcome one or more of the above-described drawbacks and/or disadvantages of the prior art.

### SUMMARY OF THE INVENTION

In accordance with a first aspect, the present invention is directed to a device for detecting nucleic acids in a biological sample. The device comprises a sample port for receiving therein a biological sample; a solid-state membrane configured to capture nucleic acids in the biological sample passed across the membrane; a sample conduit in fluid communication between the sample port and the solid-state membrane; a lysis station in fluid communication with the sample conduit and including a lysis agent therein; a wash station in fluid communication with at least one of the sample conduit or the solid-state membrane and including a wash solution therein; an elution station in fluid communication with at least one of the sample conduit or the solid-state membrane and including an eluent therein; a waste chamber located downstream of the solid-state membrane; and one or more reaction chambers located downstream of the solid-state membrane. The sample port, lysis station and sample conduit are configured to mix the sample and lysis agent to form a sample-lysis mixture, pass the sample-lysis mixture across the solid-state membrane to capture nucleic acids in the biological sample therein, and receive the remainder of the sample-lysis mixture in the waste chamber. The wash station is configured to introduce the wash solution into at least one of the sample conduit or solid-state membrane following the sample-lysis mixture to purify nucleic acids captured on the solid-state membrane. The wash solution from the solid-state membrane is received in the waste chamber. The elution station is configured to pass the eluent across the solid-state membrane, elute captured nucleic acids from the solid-state membrane, and pass the captured nucleic acids into one or more reaction chambers configured for amplifying and detecting the captured nucleic acids therein.

Some embodiments of the present invention further comprise (i) a lysis leg extending in fluid communication between the lysis station and the sample conduit and configured to direct a flow of the lysis agent from the lysis station into the sample conduit, and (ii) a wash leg extending in fluid communication between the wash station and the sample conduit at a point upstream relative to the lysis leg and configured to direct a flow of the wash solution from the wash station into the sample conduit behind the sample-lysis mixture. In some such embodiments, (i) the wash leg is in fluid communication with the sample conduit at a sample-wash junction located adjacent to the sample port and configured to allow a substantial portion of the sample to flow into the sample conduit downstream of the sample-wash junction prior to introducing the wash solution through the wash leg and into the sample conduit; and (ii) the lysis leg is in fluid communication with the sample conduit at a sample-lysis junction located downstream of the sample-wash junction and configured to allow the lysis agent to mix with the sample and form the sample-lysis mixture and the wash solution to flow into the sample conduit behind or upstream of the sample-lysis mixture.

Some embodiments of the present invention further comprise a static mixer in fluid communication between the sample-lysis junction and the solid-state membrane to mix the sample and lysis agent and form a sample-lysis mixture prior to passage across the solid-state membrane. In some such embodiments, the static mixer is defined by a plurality of axially-spaced recesses or grooves formed in the sample conduit. In some embodiments, the axially-spaced recesses or grooves are formed in a bottom wall of the sample conduit and are shaped such that a first part of each groove imparts a first rotational component to a fluid flowing therethrough in a first direction, and a second part of each groove imparts a second rotational component to the fluid flowing therethrough in a second direction that is different than the first direction, to thereby facilitate mixing the sample and lysis agent into the sample-lysis mixture. In some such embodiments, the first part of each recess or groove is oriented approximately at an acute angle relative to the second part of each recess or groove. In some embodiments, the first part of each recess or groove is shorter than then second part of each recess or groove. In some such embodiments, the plurality of axially-spaced recesses or grooves define an approximate herringbone shape.

In some embodiments of the present invention the lysis station includes a sealed lysis agent chamber containing the lysis agent, and a lysis actuator movable between a non-actuated position and an actuated position. In the actuated position the lysis agent is released from the lysis chamber through the lysis leg and into the sample conduit where the lysis agent is mixed with the sample into the sample-lysis mixture. In some such embodiments, the wash station includes a sealed wash chamber containing the wash solution, and a wash actuator movable between a non-actuated position and an actuated position. In the actuated position the wash solution is released from the wash chamber through the wash leg and into the sample conduit behind or upstream of the sample-lysis mixture. In some such embodiments, the lysis actuator is manually actuated and movable from the non-actuated position to the actuated position, and the lysis chamber includes a frangible or breakable wall that is breakable by movement of the lysis actuator in the actuated position to release the lysis agent from the lysis chamber into the lysis leg. In some such embodiments, the wash actuator is manually actuated and movable from the non-actuated position to the actuated position, and the wash chamber includes a frangible or breakable wall that is breakable by movement of the wash actuator in the actuated position to release the wash solution from the wash chamber into the wash leg.

In some embodiments of the preset invention the lysis actuator is a lysis plunger and the lysis chamber includes a lysis blister containing the lysis agent therein. Movement of the lysis plunger from the non-actuated to the actuated position causes the lysis plunger to break the lysis blister and release the lysis agent into the lysis leg. In some such embodiments, the wash actuator is a wash plunger and the wash chamber includes a wash blister containing the wash solution therein. Movement of the wash plunger from the non-actuated to the actuated position causes the wash plunger to break the wash blister and release the wash solution into the wash leg.

Some embodiments of the present invention further comprise a second wash station in fluid communication with the sample conduit that includes a second wash solution therein. The second wash station is configured to introduce the second wash solution into the sample conduit following the other wash solution and to pass the second wash solution across the solid-state membrane to purify nucleic acids captured therein. The second wash solution passes across the solid-state membrane and is received in the waste chamber.

In some embodiments of the present invention, the second wash station includes a sealed second wash chamber containing the second wash solution. A second wash leg is in fluid communication between the second wash station and the sample conduit downstream of the other wash leg and is configured to direct a flow of the second wash solution from the second wash station into the sample conduit. A second wash actuator is movable between a non-actuated position and an actuated position. In the actuated position the second wash solution is released from the second wash chamber through the second wash leg and into the sample conduit, is passed across the solid-state membrane to purify nucleic acids captured therein, and is received in the waste chamber. In some embodiments of the present invention, the second wash actuator is manually actuated and movable from the non-actuated position to the actuated position, and the second wash chamber includes a frangible or breakable wall that is breakable by movement of the second wash actuator in the actuated position to release the second wash solution from the second wash chamber into the second wash leg. In some such embodiments, the second wash actuator is a plunger and the second wash chamber includes a second wash blister containing the second wash solution therein. Movement of the plunger from the non-actuated position to the actuated position causes the plunger to break the second wash blister and release the second wash solution into the second wash leg.

In some embodiments of the present invention, the elution station includes a sealed eluent chamber containing the eluent, an elution leg extending in fluid communication between the elution station and the solid-state membrane, and an elution actuator movable between a non-actuated position and an actuated position. In the actuated position the eluent is released from the eluent chamber through the elution leg and across the solid-state membrane to elute captured nucleic acids from the solid-state membrane and pass the captured nucleic acids into the reaction chamber. In some such embodiments, the elution actuator is manually actuated and movable from the non-actuated position to the actuated position, and the eluent chamber includes a frangible or breakable wall that is breakable by movement of the elution actuator in the actuated position to release the eluent from the eluent chamber into the elution leg. In some embodiments, the elution actuator is a plunger and the eluent chamber includes an eluent blister containing the eluent therein, and movement of the plunger from the non-actuated to the actuated position causes the plunger to break the frangible or breakable wall and release the eluent into the elution leg.

Some embodiments of the present invention further comprise a lysis-wash actuator fixedly connected to the lysis plunger and to the wash plunger and manually depressible to move the lysis plunger and the wash plunger from the non-actuated position to the actuated position. Some such embodiments further comprise a plunger mount connected to the lysis-wash actuator, a plunger spring defining a plunger spring force and mounted between the lysis-wash actuator and the plunger mount, and a latch. Upon manually moving the lysis-wash actuator from a non-actuated position to a first actuated position, the latch secures the lysis-wash actuator in the first actuated position where the lysis plunger engages the lysis blister and the wash plunger engages the wash blister, and the plunger spring drives the plunger mount from the first actuated position to a second actuated position. The lysis plunger and wash plunger break the frangible or breakable walls of the lysis agent chamber and the wash solution chamber, respectively, and the plunger spring drives the lysis plunger and the wash plunger into the respective chambers to release the lysis agent and the wash solution into the lysis leg and wash leg, respectively. In some such embodiments, the latch includes a locking tab and a latch spring coupled to the locking tab. During movement of the lysis-wash actuator between the non-actuated positon and the first actuated position, the latch spring urges the locking tab into contact with the lysis-wash actuator but allows relative movement thereof. In the first actuated position, the latch spring urges the locking tab into a locked position securing the lysis-wash actuator in the first actuated position.

Some embodiments of the present invention further comprise a base station for receiving the device. The base station includes a lock-release bar moveable between (i) a locked position where it prevents movement of the lysis-wash actuator and (ii) a release position where it allows movement of the lysis-wash actuator into the first actuated position. The device is movable relative to the base station between a non-operational position and an operational position. During movement of the device between the non-operational position and the operational position, the lock-release bar is moved from the locked position to the release position to thereby allow the lysis-wash actuator to move into the first actuated position when the device is located in the operational position. Some embodiments further comprise a lock-release spring coupled to the lock release bar and urging the lock release bar in a direction from the release position to the locked position. Upon removal of the device from the base station, the lock-release spring drives the lock-release bar from the release positon to the locked position, and the lock-release bar drives the lysis-wash actuator upwardly from the first actuated positon to the non-actuated position.

Some embodiments of the present invention further comprise a base station for receiving the device. The base station includes a ramp, a sled mounted on the ramp, and a heater mounted on the sled. Upon receiving the device into the base station, the sled is connectable to the device and movable therewith between a non-operational position and an operational position. During movement between the non-operational position and the operational position, the heater is moved from a non-operational positon out of contact with the device to an operational position in contact with the device and adjacent to the reaction chamber for incubating the captured nucleic acids within the reaction chamber. In some embodiments, the reaction chamber(s) are preheated by activating the heater prior to filling the reaction chamber(s), such as upon movement of the heater into the operational position. Some embodiments of the present invention further comprise a heater spring mounted between the sled and the heater and urging the heater into contact with the device in the operational position. In some embodiments, the sled includes a tang on a distal end thereof, and the device includes a connecting recess on a distal end thereof. Upon inserting the device into the base station, the tang is partially received in the connecting recess. During movement between the non-operational position and the operational position, the tang is more fully received in the connecting recess. In some embodiments, the base station includes a spring-biased latch engageable with the connecting recess in the operational position to releasably retain the device in the operational positon.

Some embodiments of the present invention further comprise a waste chamber vent in fluid communication between the waste chamber and ambient atmosphere. The waste chamber vent defines an open condition and a closed condition. In the open condition fluid passing across the solid-state membrane is received within the waste chamber. In the closed condition fluid passing across the solid-state member is prevented from passing into the waste chamber. In some such embodiments, during passage of the sample-lysis mixture and wash solution across the solid-state membrane, the waste chamber vent is in the open condition and the sample-lysis mixture and the wash solution passing across the solid-state membrane flow into the waste chamber and are prevented from flowing into the reaction chamber.

In some embodiments of the present invention, the elution station includes a first sealed eluent chamber containing a first eluent; a first elution leg in fluid communication between the first elution station and the solid-state membrane; a second sealed eluent chamber containing a second eluent; and a second elution leg in fluid communication between the second elution station and the solid-state membrane. An elution actuator is movable between a non-actuated position and an actuated position. In the actuated position the first and second eluents are released from the first and second eluent chambers and into the first and second elution legs. In some such embodiments, the elution actuator substantially simultaneously releases the first and second eluents from the first and second eluent chambers, and the second elution leg is longer than the first elution leg to thereby allow the first eluent to pass across the solid-state membrane prior to passage of the second eluent across the solid-state membrane.

In some embodiments of the present invention, the wash solution and/or lysis agent leaves an evaporative contaminant on the solid-state membrane after passage therethrough. The first elution leg contains a volume of air or other gas therein such that upon releasing the first eluent into the first elution leg, the volume of air or other gas in the first elution leg is passed across the solid-state membrane and substantially evaporates the evaporative contaminant and thereby prevents contamination of the first eluent and captured nucleic acids received within the reaction chamber. In some such embodiments, the evaporative contaminant is ethanol, and the volume of air or other gas in the first elution leg is sufficient to substantially evaporate the ethanol in and about the solid-state membrane prior to passage of the first eluent across the membrane to thereby prevent ethanol poisoning of the reaction chamber.

In some embodiments of the present invention, the elution actuator is manually actuated and movable from the non-actuated position to the actuated position. Each of the first and second eluent chambers includes a frangible or breakable wall that is breakable by movement of the elution actuator in the actuated position to release the first and second eluents into the first and second elution legs, respectively. In some such embodiments, movement of the elution actuator into the actuated position partially dispenses the first and second eluents into the first and second elution legs, respectively. During such partial dispensing, the waste chamber vent is in the open condition to thereby allow any wash solution and/or air with evaporative contaminants in or about the solid-state membrane to flow into the waste chamber and not into the reaction chamber. After partially dispensing the first and second eluents and flowing any remaining wash solution in or about the solid-state membrane into the waste chamber, the waste vent is in the closed condition to thereby direct the first eluent and captured nucleic acids from the solid-state membrane into the reaction chamber.

Some embodiments of the present invention comprise a waste vent seal movable between (i) an open position allowing fluid to flow out of the waste chamber vent and thereby allow fluid to flow into the waste chamber, and (ii) a closed position sealing the vent and thereby preventing fluid from flowing into the waste chamber. In some such embodiments, the waste vent seal is mounted on the elution actuator. In some embodiments, the elution actuator includes a manually-engageable portion, a plunger mount, a first elution plunger engageable with the first eluent chamber, a second elution plunger engageable with the second eluent chamber, a plunger spring defining a plunger spring force and mounted between the manually-engageable portion and the plunger mount, and a latch. Upon manually moving the manually-engageable portion from a non-actuated position to a first actuated position, the latch secures the manually-engageable portion in the first actuated position where the first elution plunger partially dispenses the first eluent chamber and the second elution plunger partially dispenses the second eluent chamber. Then, the plunger spring drives the plunger mount from the first actuated position to a second actuated position under the plunger spring force to further dispense the first and second eluents from the first and second eluent chambers, respectively. Some embodiments further comprise a waste vent seal spring urging the waste vent seal in a direction from the open position to the closed position. In some such embodiments, the waste vent seal is mounted on the plunger mount, and the waste vent seal spring is mounted between the waste vent seal and the plunger mount. Upon movement of the manually-engageable portion into the first actuated position, the plunger spring and the waste vent seal spring urge the waste vent seal into the closed position to thereby seal the waste chamber vent.

Some embodiments of the present invention further comprise a reaction chamber valve in fluid communication between the solid-state membrane and the reaction chamber. The reaction chamber valve (i) is closed to prevent fluid flow into the reaction chamber when a fluid pressure between the solid-state membrane and the reaction chamber valve is below a valve-opening pressure, or (ii) is open to allow fluid flow into the reaction chamber when the fluid pressure between the solid-state membrane and the reaction chamber valve is above the valve-opening pressure. In some embodiments, movement of the waste chamber vent seal into the closed position causes the fluid pressure between the solid-state membrane and reaction chamber valve to exceed the valve-opening pressure and thereby allow fluid flow from the solid-state membrane into the reaction chamber.

Some embodiments of the present invention further comprise a first reaction chamber and a first reaction chamber valve in fluid communication between the solid-state membrane and the first reaction chamber. The first reaction chamber valve defines a first valve-opening pressure. The device further comprises a second reaction chamber and a second reaction chamber valve in fluid communication between the solid-state membrane and the second reaction chamber. The second reaction chamber valve defines a second valve-opening pressure that is greater than the first valve opening pressure, to thereby cause the eluted nucleic acids to first flow into the first reaction chamber and then flow into the second reaction chamber. In some such embodiments, each reaction chamber valve is a Laplace, Laplace-type or burst valve.

Some embodiments of the present invention further comprise a first reaction chamber vent in fluid communication between the first reaction chamber and ambient atmosphere. The first reaction chamber vent allows gas but substantially prevents liquid flow therethrough, to thereby flow eluted nucleic acids into the second reaction chamber after filling the first reaction chamber. Some embodiments further comprise a second reaction chamber vent in fluid communication between the second reaction chamber and ambient atmosphere. The second reaction chamber vent allows gas but substantially prevents liquid flow therethrough to thereby prevent liquid from flowing into the second reaction chamber upon filling the second reaction chamber with liquid. In some such embodiments, each of the first and second reaction chamber vents includes a hydrophobic vent membrane that allows gas but substantially prevents liquid flow therethrough. Some embodiments further comprise (i) a first reconstitution chamber in fluid communication between the first reaction chamber valve and the first reaction chamber, and (ii) a second reconstitution chamber in fluid communication between the second reaction chamber valve and the second reaction chamber.

Some embodiments of the present invention comprise a plurality of actuators. Each actuator is manually movable from a non-actuated position to an actuated position. Each of the lysis station, wash station and elution station includes a sealed chamber including a frangible or breakable wall and containing therein the lysis agent, wash solution or eluent, respectively. Upon movement of each actuator from the non-actuated to the actuated position, one or more of the frangible or breakable walls is broken to release at least one of the lysis agent, wash solution and/or eluent from its respective sealed chamber. In some embodiments, one or more of the actuators includes a plunger engageable with a respective sealed chamber. The plunger defines an axial direction of movement and includes first and second chamber-engaging surfaces. The first chamber-engaging surface defines a first width or diameter. The second chamber-engaging surface extends outwardly relative to the first chamber-engaging surface in the axial direction of movement and defines a second width or diameter that is less than the first width or diameter. Movement of the actuator from the non-actuated to the actuated positon moves the second chamber-engaging surface into engagement with the respective sealed chamber and breaks the frangible or breakable wall. In some embodiments the first chamber-engaging surface defines a first diameter, and the second chamber-engaging surface defines a second diameter, wherein the second diameter is less than the first diameter. In some such embodiments, the second diameter is at least about two times and preferably about three times less than the first diameter. In some embodiments, the first chamber-engaging surface is defined by at least one first radius of curvature, the second chamber-engaging surface is defined by at least one second radius of curvature, and the least one first radius of curvature is greater than the at least one second radius of curvature. In some embodiments, each of the lysis station, wash station and elution station includes a piercing member engageable with the respective frangible or breakable wall upon moving a respective actuator from the non-actuated position to the actuated position. The piercing member facilitates breaking the wall, and each second chamber-engaging surface includes a recess therein for receiving the piercing member at least partially therein. In some embodiments, each of the lysis station, wash station and elution station includes a recessed surface located below the respective sealed chamber for receiving the lysis agent, wash solution or eluent upon breaking the respective wall. An outlet is located at approximately the lowest point of the recessed surface and is in fluid communication with the lysis leg, wash leg or elution leg, respectively. In some embodiments, each recessed surface defines a plurality of elongated grooves therein in fluid communication with the outlet and angularly spaced relative to each other to facilitate the flow of fluid into the outlet.

In some embodiments of the present invention, the solid-state membrane includes an inlet side and an outlet side. The device further comprises a membrane inlet located on the inlet side of the solid-state membrane and in fluid communication between the sample conduit and elution station, on the one hand, and the solid-state membrane, on the other hand. A membrane outlet is located on the outlet side of the solid-state membrane in fluid communication between the solid-state membrane and the waste chamber or reaction chamber. The membrane inlet defines a plurality of inlet fluid channels configured to facilitate a flow of fluid across the inlet side of the solid-state membrane, and the membrane outlet includes a plurality of fluid outlet channels therein configured to facilitate a flow of fluid across the outlet side of the solid-state membrane. In some such embodiments, the inlet fluid channels include a plurality of radially-extending inlet fluid channels angularly spaced relative to each other, and the outlet fluid channels include a plurality of radially-extending outlet fluid channels angularly spaced relative to each other. In some embodiments, the inlet fluid channels include at least one annularly extending inlet fluid channel intersecting at least a plurality of the radially-extending inlet fluid channels, and the outlet fluid channels include at least one annularly extending outlet fluid channel intersecting at least a plurality of the radially-extending outlet fluid channels.

Some embodiments of the present invention further comprise a body including the at least one reaction chamber therein. At least a portion of the body including the reaction chamber(s) is substantially transparent, and includes a substantially transparent top surface extending over the reaction chamber(s) and two substantially transparent side surfaces extending downwardly from the top surface along opposite sides of the reaction chamber(s) relative to each other. The reaction chamber(s) is visually observable in a viewing direction through the substantially transparent top surface. A stimulating light source is located adjacent to a substantially transparent side surface configured to transmit stimulating light through the side surface and reaction chamber(s) in a direction substantially lateral to the viewing direction. In some embodiments, the index of refraction of the substantially transparent body and the index of refraction of the fluid in the reaction chamber(s) are configured to facilitate the passage of the stimulating light from the body into the reaction chamber(s) to generate fluorescing light in the reaction chamber. The fluorescing light is emitted in substantially all directions and is observable in the viewing direction through the top surface of the body. Preferably, there is an observable difference to the human eye between the stimulating light and the fluorescing light to facilitate the ability of an observer to view the fluorescing light and distinguish it from any observed stimulating light. In some embodiments, the stimulating light defines a first wavelength, and the fluorescing light defines a second wavelength, wherein the first wavelength is less than the second wavelength. In some such embodiments, the first wavelength is within the range of about 425 nm to about 550 nm. In some embodiments, the first wavelength is about 470 nm and the second wavelength is about 510 nm. In some embodiments of the invention, the top surface of the body is substantially smooth or polished and the side surfaces of the body are substantially smooth or polished to facilitate maintaining the stimulating light within the substantially transparent body.

In some embodiments of the invention, the body is substantially transparent throughout. The body defines integrally molded therein the sample conduit, a lysis leg extending in fluid communication between the lysis station and the sample conduit and configured to direct a flow of the lysis agent from the lysis station into the sample conduit, a wash leg extending in fluid communication between the wash station and the sample conduit at a point upstream of the lysis leg and configured to direct a flow of the wash solution from the wash station into the sample conduit behind the sample-lysis mixture, an elution leg extending in fluid communication between the elution station and the solid-state membrane, the reaction chamber and the waste chamber.

Some embodiments of the present invention further comprise a saliva collection swab for collecting saliva thereon and receivable within the sample port for introducing the saliva directly into the sample port and sample conduit for mixture therein with the lysis agent. In some such embodiments, the saliva collection swab includes a plunger depressible against the saliva collection swab within the sample port to release saliva from the collection swab into the sample port and sample conduit. In some such embodiments, at least one of the saliva collection swab or the sample port includes a locking tab, and the other of the saliva collection swab or sample port includes a corresponding locking recess or aperture configured to receive the locking tab and retain the swab within the sample port with the plunger depressed against the swab to facilitate release of saliva therefrom and into the sample port.

Some embodiments of the present invention further comprise a reaction chamber vent in fluid communication between the reaction chamber and ambient atmosphere. The reaction chamber vent defines a venting length extending between the reaction chamber and ambient atmosphere and a venting cross-sectional area. The venting cross-sectional area is sufficiently small compared to the venting length to create a saturation gradient between the reaction chamber and ambient atmosphere to slow the evaporation of liquid from, and prevent the entry of atmospheric air into, the reaction chamber.

Some embodiments of the present invention further comprise a body defining therein the sample conduit, a lysis leg extending in fluid communication between the lysis station and the sample conduit and configured to direct a flow of the lysis agent from the lysis station into the sample conduit, a wash leg extending in fluid communication between the wash station and the sample conduit upstream of the lysis leg and configured to direct a flow of the wash solution from the wash station into the sample conduit behind the sample-lysis mixture, an elution leg extending in fluid communication between the elution station and the solid-state membrane, and a viewing window overlying the reaction chamber and allowing visual observation of the reaction chamber therethrough in a viewing direction. A heater is mounted to the body adjacent to the reaction chamber and configured to heat the reaction chamber. A stimulating light source is configured to transmit stimulating light into the reaction chamber in a direction lateral to the viewing direction. A power source, such as a battery, a plug, or a receptacle, is connected to the heater and light source and configured to provide power thereto.

In accordance with another aspect, the present invention is directed to a method for detecting nucleic acids in a biological sample. The method comprises:

receiving a biological sample through a sample port and into a sample conduit in fluid communication between the sample port and a solid-state membrane for capturing nucleic acids in the biological sample and amplifying and detecting the captured nucleic acids therein in at least one reaction chamber;

introducing a lysing agent into the sample conduit, mixing the lysing agent with the sample to form a sample-lysis mixture, passing the sample-lysis mixture across the solid-state membrane and capturing nucleic acids in the biological sample therein, preventing the flow of the sample-lysis mixture that passes across the solid-state membrane into the at least one reaction chamber, and receiving the remainder of the sample-lysis mixture that passes across the solid-state membrane in a waste chamber;

introducing a wash solution into at least one of the sample conduit or solid-state membrane following the sample-lysis mixture, passing the wash solution across the solid-state membrane and purifying nucleic acids captured from the sample-lysis mixture therein, preventing the flow of the wash solution into the reaction chamber, and receiving the wash solution that passes through the solid-state membrane in the waste chamber; and

introducing an eluent across the solid-state membrane and eluting captured nucleic acids from the solid-state membrane, substantially preventing the captured nucleic acids from flowing into the waste chamber, directing the captured nucleic acids into the at least one reaction chamber, and amplifying and detecting the captured nucleic acids in the at least one reaction chamber.

Some embodiments of the present invention further comprise introducing a second wash solution into the sample conduit following the other wash solution and passing the second wash solution across the solid-state membrane and purifying nucleic acids captured therein, preventing the flow of the second wash solution into the reaction chamber, and receiving the second wash solution that passes through the solid-state membrane in the waste chamber.

Some embodiments of the present invention further comprise closing a vent to the waste chamber after receiving the lysis agent and wash solution therein, and opening an inlet valve to the reaction chamber(s) for directing the captured nucleic acids from the solid-state membrane therein.

In some embodiments, the wash solution and/or lysis agent leaves an evaporative contaminant on the solid-state membrane after passage therethrough, and the method further comprises flowing air or other gas across the solid-state membrane prior to passage of the eluent therethrough and substantially evaporating the evaporative contaminant and preventing contamination of the eluent and captured nucleic acids.

In some embodiments each reaction chamber defines a viewing direction for visually observing an interior thereof, and the method further comprises directing stimulating light into the reaction chamber lateral to the viewing direction, generating fluorescing light in substantially all directions and allowing for visual observation in the viewing direction a greater amount of the fluorescing light as compared to the stimulating light.

One advantage of the invention, and/or of embodiments thereof, is that the sample-lysis mixture and wash solution or buffers are not introduced into the reaction chamber. Rather, they bypass the reaction chamber and are introduced into the separate waste chamber. As a result, the volumes of the biological sample, lysate mixture and wash buffers are not limited by the capacity of the reaction chamber, as in the above-described prior art. Yet another advantage is that the need to encapsulate the dry reagents in a low-melting point paraffin and the associated drawbacks thereof can be avoided. As a result, the device and method of the invention can allow for greater volumes of biological samples to be passed across the membrane, and greater amounts of targeted nucleic acids to be captured, purified and amplified. This can, in turn, lead to a more sensitive detection of, and testing for, targeted nucleic acids.

Yet another advantage of the invention, and/or of embodiments thereof, is that less force may be required to depress the blisters and rupture the flexible lidding film or other breakable or frangible walls than in the above-described prior art. For example, the manually-engageable actuators or plungers may include the first and second chamber-engaging surfaces, where the second chamber-engaging surface defines a smaller width or diameter than the first chamber-engaging surface. These features may take the form of a “bump on a bump” configuration. A significant advantage is that these features can reduce the manual force required to depress the blisters and rupture the films or other frangible or breakable walls in order to release the liquids therefrom, as compared to the above-described prior art.

A still further advantage of the invention, and/or of embodiments thereof, is that the elution leg of the elution chamber can contain a sufficient volume or air or other gas that is passed over the solid-state membrane prior to passage of the eluent therethrough. As a result, substantially all residual ethanol can be evaporated by the air or other gas and the ethanol poisoning encountered in the above-described prior art can be avoided. Yet another advantage is that this configuration can be implemented in a lab on chip system or device.

A still further advantage of the invention, and/or of embodiments thereof, is that the use of sharp cutoff or passband filters to suppress the observation of the stimulating light, and their associated costs, can be avoided. Rather, the stimulating light may be transmitted through the substantially transparent body in a direction substantially lateral or perpendicular to the direction of viewing the reaction chamber(s), thus geometrically separating the different lights and facilitating the ability to visually observe only, or primarily, the fluorescing light.

Yet another advantage of the invention, and/or of embodiments thereof, is that the saliva or other biological sample is inserted into the sample port to seal the sample port and deposit the saliva or other biological sample directly into the sample conduit where it is mixed with a lysing agent prior to passage across the solid-state membrane. As a result, the relatively complex processing of samples, and the associated risks and disadvantages thereof, as encountered in the above-described prior art can be avoided.

Still another advantage of the invention, and/or of embodiments thereof, is that preheating the reaction chamber or chambers can cause the reaction to occur more quickly and/or and reduce the time required for the reaction to complete. As a result, in a clinical setting, the number of tests that may be performed in a given time period can be increased and, in any setting, the wait time for a patient or user to receive results can be reduced.

Other advantages of the present invention, and/or of the embodiments thereof, will become more readily apparent in view of the following detailed description and accompanying drawings.

### BRIEF DESCRIPTION OF THE DRAWINGS

FIG. 1 includes eight perspective views of a device embodying the present invention including a disposable cartridge received within a base station for detecting nucleic acids in a biological sample, where the different views are labeled “1” through “8” and progressively show the operation of the device, view “1” shows the cartridge prior to insertion into the base station, view “2” shows the cartridge inserted into the base station, and the views thereafter show the procedural steps of progressively depressing the buttons on the base station such that the device performs the method of detecting nucleic acids in the biological sample;

FIGS. 2A and 2B are perspective views of a sample syringe for biological sample collection and insertion into the inlet port of the disposable cartridge of FIG. 1 for introducing a saliva sample into the sample port and sample conduit, and including in FIG. 2A a view of the sample syringe not inserted into the inlet port of the cartridge and in FIG. 2B a view of the sample syringe inserted into the inlet port of the cartridge and locked therein by the locking tabs received in the corresponding locking apertures of the sample port;

FIG. 3 is a perspective view of the disposable cartridge and sample syringe of FIG. 1 showing the sample syringe prior to insertion into the sample port of the cartridge;

FIG. 4 is a perspective view of the underside of the disposable cartridge of FIG. 1 showing the recesses for the various stations and microfluidic channels formed therein;

FIG. 5 is a plan view of the underside of the disposable cartridge of FIG. 4;

FIG. 6A is a perspective view of the underside of the chassis of the disposable cartridge of FIG. 1 and FIG. 6B is perspective view of the top of the chassis of the disposable cartridge, showing the recesses formed therein for injection molding purposes, the vent pathways, and the Laplace valves;

FIG. 7A is a perspective view of the underside of the chassis of the disposable cartridge of FIG. 1 and FIG. 7B is a perspective view of the top of the chassis of the disposable cartridge, showing the vent condition when the cartridge is initially inserted into the base station, wherein the vent to the waste chamber is open and the vents to the reaction chambers are closed, in order to allow fluid to flow to the waste chamber and prevent fluid from flowing to the reconstitution and reaction chambers;

FIGS. 8A and 8B include the perspective views of the chassis of FIGS. 7A and 7B, showing a saliva sample injected into the inlet port and sample conduit;

FIGS. 9A and 9B include the perspective views of the chassis of FIGS. 8A and 8B, showing in FIG. 9A the saliva sample injected into the inlet port and sample conduit and the “dead leg” or air pocket created by the wash leg extending between the first wash station “1” and the sample conduit to separate the first wash solution from the sample and the sample-lysis agent mixture, and showing in FIG. 9B that the blisters of the first wash station “1” and the lysis station “2” are pressed and punctured, the vent to the waste chamber is open and the vents to the reaction chambers are closed;

FIGS. 10A and 10B include the perspective views of the chassis of FIGS. 9A and 9B, showing in FIG. 10A that continued dispensing from the blisters of the first wash station “1” (red) and the lysis station “2” (yellow) causes the sample and lysis agent to gradually mix in the static mixer into the sample-lysis agent mixture, and the first wash solution follows the sample-lysis agent mixture in the sample conduit, and further showing the sample-lysis agent mixture passing across the solid-state membrane and into the waste sump or chamber;

FIGS. 11A through 11C include in FIG. 11A a partial, perspective view of the chassis of FIG. 1, and in FIGS. 11B and 11C, progressively enlarged, partial, perspective views of the static or herringbone mixer formed in the bottom wall of the sample conduit for mixing the sample and lysis agent into the sample-lysis agent mixture as it flows therethrough;

FIGS. 12A and 12B include the perspective views of the chassis of FIGS. 10A and 10B, and show the liquid conditions when the blisters of the first wash station “1” and the lysis station “2” are fully dispensed and the blister of the second wash station “3” containing the second wash solution (white) is initially pressed such that the second wash solution (white) flows into the second wash leg prior to being introduced in the sample conduit behind the first wash solution (with an air pocket from the second wash leg therebetween);

FIGS. 13A and 13B include the perspective views of the chassis of FIG. 12A and 12B, and show the liquid conditions when the blisters of the first wash station “1” and lysis station “2” are fully dispensed and the blister of the second wash station “3” continues to dispense, wherein the sample-lysis mixture (brown) is passed across the solid-state membrane and received within the waste sump, and the second wash solution (white) flows into the sample conduit and across the solid-state membrane behind the first wash solution and into the waste sump;

FIGS. 14A and 14B include the perspective views of the chassis of FIGS. 13A and 13B, and show the liquid conditions when blisters of the first wash station “1” and lysis station “2” are fully dispensed, and blister of the second wash station “3” continues to dispense the second wash solution (white) to completion, wherein the second wash solution passes across the solid-state membrane and into the waste sump;

FIGS. 15A and 15B include the perspective views of the chassis of FIGS. 14A and 14B, and show the liquid conditions when the blisters of the elution stations “4” and “5” are partially depressed and partially dispensed, and the initial condition of the valve during such partial dispensing, where the vent to waste sump is open and the vent to reaction chambers is closed, in order to allow the “dead leg” of air within the elution leg of station “5” to push the remaining second solution (white) through the solid-state membrane and the air to, in turn, dry the solid-state membrane and substantially evaporate any ethanol in or about the membrane;

FIGS. 16A and 16B include the perspective views of the chassis of FIGS. 15A and 15B, and show the liquid conditions when the elution stations “4” and “5” are partially dispensed and the vents are flipped, i.e., the waste sump vent is closed and the reaction chamber vents are open, in order to divert the flow of eluted nucleic acids from the solid-state membrane to the reconstitution and reaction chambers;

FIGS. 17A and 17B include the perspective views of the chassis of FIGS. 16A and 16B, and show the liquid conditions when the blisters of the elution stations “4” and “5” continue to be dispensed, wherein the negative control (green) bypasses the solid-state membrane and flows into its respective negative control reconstitution and reaction chambers, and the eluent from station “5” (gray) flows across the solid-state membrane and is initially directed into a first reconstitution chamber and reaction chamber, and then after filling the first reaction chamber, is directed into a second reconstitution and reaction chamber;

FIGS. 18A through 18F show a series of side, partial cross-sectional views of the process of inserting the disposable cartridge into the base station, and include partial, magnified views in FIGS. 18B and 18F, showing the distal end of the cartridge releasably engaging the spring-biased latch in the housing in the operational position, and the heater of the housing spring-biased into engagement with the underside of the cartridge adjacent to the reaction chambers to heat the reaction chambers and thereby incubate the reaction;

FIGS. 19A and 19B include two somewhat schematic, side views of the disposable cartridge of FIG. 1 (or chip assembly) received within and engaged with the sled in the base station, wherein further insertion of the cartridge into the base station (FIG. 19B) causes the sled to ride up the ramps, and the spring-biased heater moves into engagement with the underside of the cartridge adjacent to the reaction chambers in the fully-inserted or operational position;

FIG. 20A is an upper perspective view, and FIG. 20B is a top plan, somewhat schematic view of the flexible circuit mounted within the base station and including light emitting diodes that transmit stimulating light transversely into the transparent chassis and into the reaction chambers when the disposable cartridge is fully received in the operational position in the base station;

FIG. 21 is partial, top plan view of the chassis fully received in the operational position in the base station with the reaction chambers aligned with the light emitting diodes;

FIG. 22 is a perspective view of the cartridge chassis overlying the flex circuit and located adjacent to the printed circuit board of the base station when fully inserted into the base station in the operational position;

FIG. 23 is a somewhat schematic view of the distal end portion of the chassis of the cartridge of FIG. 1 showing with arrows the stimulating light transmitted by the LEDs transversely through the side wall of the chassis and into the reaction chambers and the resulting fluorescing light emitted in all directions, such that the fluorescing light is visible in the viewing direction through the top wall of the chassis and the stimulating light is geometrically separated from the fluorescing light and is substantially not visible in the viewing direction;

FIGS. 24A through 24C show three cross-sectional views of a plunger of the disposable cartridge of FIG. 1 defining a bump on a bump configuration including a first blister-engaging surface, and a second smaller diameter blister-engaging surface projecting downwardly from the first blister-engaging surface, wherein the smaller diameter blister-engaging surface initially engages the blister (FIGS. 24A and 24B) and thereby reduces the downward force required to depress and break the underlying frangible or breakable wall (FIG. 24C);

FIGS. 25A and 25B include two somewhat schematic views of the plunger of FIGS. 24A-24C showing the progressive movement of the plunger and its first and second blister-engaging surfaces into engagement with the blister, and as shown in FIG. 25B, the breaking of the underlying frangible or breakable wall upon contacting the underlying rupture pin or piercing member, and the releasing of the liquid therefrom into the outlet;

FIG. 26 is a partial, cross-sectional view of the plunger mount and plungers of a button or manually-engageable actuator of the elution stations of the disposable cartridge of FIG. 1 including two plungers and a seal for the waste chamber vent, wherein pressing the button moves the two plungers into initial engagement with the blisters of the first and second elution stations, and the plunger spring then drives the plungers further into the blisters to provide a consistent plunger depressing force for dispensing the eluent therefrom, the spring-biased vent seal seals the waste chamber vent to close the waste chamber, direct the eluent and nucleic acid flow from the solid-state membrane to the reconstitution and reaction chambers, and prevent such flow into the waste chamber;

FIGS. 27A through 27E are a series of partial, cross-sectional views of the plunger mount and plungers of the button actuator of FIG. 26, showing that the button has an unactuated position and an actuated position, wherein movement of the button to the actuated position moves the plungers into contact with the blisters and causes the plunger spring to compress, such that the vent seal seals the waste chamber vent, the plunger spring further drives the plungers into the blisters to dispense substantially all of the liquid in the blisters through the outlets, and the spring-biased plungers seal the outlets in the fully actuated positions;

FIGS. 28A through 28C show a series of side views and enlarged, partial side views of the plunger mount and plungers of FIGS. 27A through 27E;

FIGS. 29A and 29B show the plungers of FIGS. 28A and 28B moving from the non-actuated position (FIG. 29A) to the fully-actuated position (FIG. 29B), wherein in the fully-actuated position, the plunger is spring-biased against the outlet to seal the outlet and prevent any backflow into the blister during subsequent processing, including during incubation of the reaction chambers;

FIGS. 30A and 30B are partial, perspective views of the cartridge of FIG. 1 showing the blisters in greater detail and an exemplary rupture pin located underneath the blister in the underlying recess of the respective station, and the angularly spaced grooves at the outlet to facilitate emptying of the released liquid therethrough;

FIGS. 31A and 31B include in FIG. 31A a partial, perspective view of the cartridge received within the base station of FIG. 1, and in FIG. 31B, a partial, perspective view of the manually-engageable actuator or button lock mechanism, wherein the lock release bar in the base station locks the buttons in a non-actuated position, and insertion of the cartridge into the base station moves the lock release bar to unlock the buttons and allow their manual actuation;

FIGS. 32A through 32E are a series of partial, perspective views of the manually-engageable actuator or button lock mechanism of FIGS. 31A and 31B showing the operation thereof;

FIG. 33 is a schematic view of a dead-leg pathway including two stations with respective blisters and associated fluid channels in fluid communication with a solid-state membrane and a waste sump, wherein the fluid channels are empty, and is representative of the operation of the lysis leg, wash leg, second wash leg and elution legs for introducing pockets of air between liquids to separate the liquids and/or for introducing air over the solid-state membrane to evaporate residual ethanol prior to passage of the eluent therethrough to prevent ethanol poisoning;

FIG. 34 is a schematic view of the dead-leg pathway in FIG. 33 after the first blister “1” has been ruptured, wherein the fluid from the first blister (red) fills the respective channel, flows through the solid-state membrane and into the waste sump;

FIG. 35 is a schematic view of the dead-leg pathway of FIGS. 33 and 34 after the second blister “2” has been ruptured, demonstrating how air in the dead-leg pushes the first fluid (red) located downstream of the dead-leg junction through the solid-state membrane and how the first fluid (to the left) and the second fluid (to the right) are separated by the air gap created by the dead leg;

FIG. 36 is a schematic view of the dead-leg pathway of FIGS. 33-35 wherein the fluid from blister “2” continues to flow filling the downstream fluid channels and without mixing with the blister “1” fluid;

FIG. 37 is a schematic view of a nest for holding the solid-state membrane of the cartridge of FIG. 1, including an inlet and an outlet for fluids to pass across the membrane;

FIG. 38 is a somewhat schematic, perspective view of the nest for receiving the solid-state membrane to facilitate distribution of liquid across the membrane without a separate device to hold the membrane;

FIG. 39 is a perspective view of a cap used with the nest of FIG. 38 to engage the other side of the membrane;

FIG. 40 is a schematic view of the Laplace valves with three holes of decreasing size that can be used in the cartridge of FIG. 1 to control progressive flow into the waste sump and then into the reaction chambers;

FIG. 41 shows a chip made from a translucent plastic and illuminated with a stimulating light showing two chambers which are stimulated by the stimulating light (speckled) and a third chamber which is not responsive to the stimulating light;

FIG. 42 is a graph showing the excitation (solid) and fluorescence (dashed) spectral responses for Calcein;

FIG. 43 is a graph showing the relative intensity of a stimulating light emitting diode (LED) at different wavelengths;

FIG. 44 is a side, somewhat schematic view of an alternate embodiment of a device embodying the present invention wherein the device does not include or require a base station, and instead the “lab on chip” device includes an integrated heater, stimulating light source and power source; and

FIG. 45 is a top plan view of the device of FIG. 45.

### DETAILED DESCRIPTION

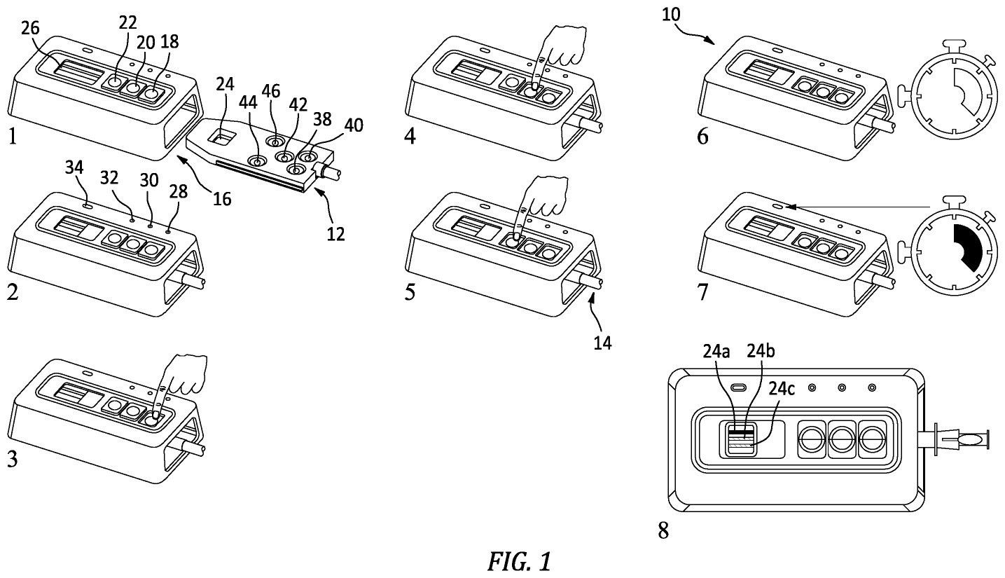

In FIG. 1, a device embodying the present invention for detecting nucleic acids in a biological sample is indicated generally by the reference numeral 10. The microfluidic test device 10 can be used to identify target nucleic acid sequences corresponding to target pathogen or host genetic sequences including any genetic target in a biological or environmental sample. Identification is done through the isolation, concentration, isothermal amplification, and detection of nucleic acids. Multiple targets can be identified simultaneously. Examples of use include without limitation: (i) the detection of infectious agents such as SARS-CoV-2 in saliva, swab, urine, or stool; (ii) the detection of specific mutations in blood samples; and (iii) the detection of infectious agents from a surface swab. The microfluidic test device 10 in FIG. 1 comprises a disposable cartridge or body 12, a sample collection device 14, and a base station 16.

The device 10 is used by placing a sample, such as saliva, into the sample collection device 14, inserting the sample collection device 14 into the body 12, and inserting the body 12 into the base station 16 before pressing a first button 18, a second button 20 and a third button 22. After waiting a period of time for a reaction to complete, the results will be visible in the reaction chamber 24 of the body 12, which can be seen through a reaction chamber window 26 in the base station 16. In at least some embodiments of the device, a first indicator 28 will indicate when to press the first button 18, a second indicator 30 will indicate when to press the second button 20, and a third indicator 32 will indicate when to press the third button 22. These indicators may be LEDs which illuminate to indicate a button should be pressed, other light sources, or other devices capable of changing color or brightness. A reaction complete indicator 34 may indicate when the reaction has completed, which in some embodiments will be approximately twenty minutes after the third button 22 is depressed (view “7” of FIG. 1).

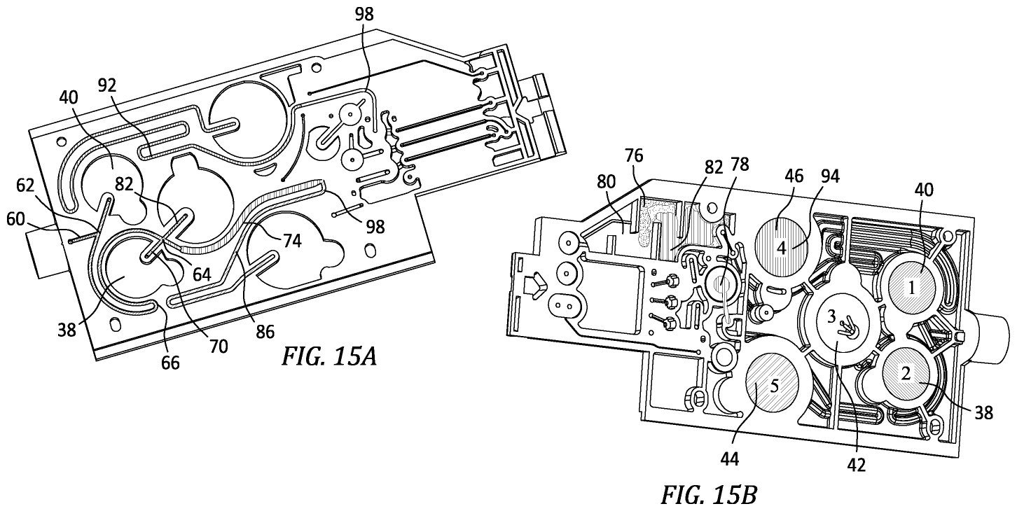

The cartridge or body 12 comprises a sample port 36, multiple microfluidic conduits, multiple stations with associated blisters 49 and manually-engageable actuators 156, a solid-state membrane 78, a plurality of reconstitution chambers 48 and associated reaction chambers 24 in fluid communication therewith, and a waste chamber or sump 80. The illustrated embodiment includes five stations (FIG. 15B): a first wash station “1”, a lysis station “2”, a second wash station “3,” an eluent station “4” and another eluent station “5.” The sample port 36 receives therein a biological sample 60, such as a saliva sample. The solid-state membrane 78 is configured to capture nucleic acids in the biological sample passed across the membrane 78. A sample conduit 35 extends in fluid communication between the sample port 36 and the solid-state membrane 78. The lysis station “2” is in fluid communication with the sample conduit 35 and includes a lysis blister 38 defining a sealed chamber 49 containing the lysis agent therein. The wash station “1” is in fluid communication with the sample conduit 35 and includes a wash solution blister 40 defining a sealed chamber 49 containing a first wash solution 66 therein. The elution station “5” is in fluid communication with the solid-state membrane 78 and includes an eluent blister 44 defining a sealed chamber 49 containing the eluent 86 therein. The other elution station “4” bypasses the solid-state membrane 78 and is in fluid communication with a respective reconstitution chamber 48a and reaction chamber 24a for providing a negative reaction control, as described further below. The waste sump or chamber 80 is located downstream of the solid-state membrane 78. A first reconstitution chamber 48 and associated reaction chamber 24 is located downstream of the solid-state membrane 78, and a second reconstitution chamber 48 and associated reaction chamber 24 is also located downstream of the solid-state membrane 78.

As described further below, the sample port 36, lysis station “2” and sample conduit 35 are configured to mix the sample 60 and lysis agent contained within a lysis buffer 70 to form a sample-lysis mixture 76, pass the sample-lysis mixture 76 across the solid-state membrane 78 to capture nucleic acids in the biological sample 60 therein, and receive the remainder of the sample-lysis mixture 76 in the waste chamber 80. The first wash station “1” is configured to introduce the first wash solution 66 into the sample conduit 35 following the sample-lysis mixture 76, pass the first wash solution 66 across the solid-state membrane 78 to purify nucleic acids captured therein, and receive the first wash solution 66 from the solid-state membrane 78 in the waste chamber 80. The second wash station “3” is configured to introduce the second wash solution 82 into the sample conduit 35 following the first wash solution 66, pass the second wash solution 82 across the solid-state membrane 78 to purify nucleic acids captured therein, and receive the second wash solution 82 from the solid-state membrane 78 in the waste chamber 80. The elution station “5” is configured to pass the eluent 86 across the solid-state membrane 78, elute captured nucleic acids from the solid-state membrane 78, and pass the captured nucleic acids initially into the first reconstitution chamber 48c and first reaction chamber 24c for amplifying and detecting the captured nucleic acids, and then into the second reconstitution chamber 48b and second reaction chamber 24b for amplifying and detecting the captured nucleic acids.

Each of the first wash blister 40, second wash blister 42, elution blister 44, and negative control blister 46 contain respective liquids as indicated above. When sufficient pressure is applied to the top 144 of each blister 49, the frangible or breakable wall of the blister bottom 146 ruptures, and the liquid therein enters the respective microfluidic conduits of the body 12, pushing the sample 60 along the sample conduit 35 and eventually pushing the eluted nucleic acids into one or more reaction chambers 24. In some embodiments of the device, there are three or more reaction chambers comprising a negative control reaction chamber 24a, a positive control reaction chamber 24b, and one or more test reaction chambers 24c. When the test is complete, the color of the test reaction chamber 24c will be compared to the color of the control chambers 24a, 24b to determine whether a positive or negative result is indicated (view “8” of FIG. 1).

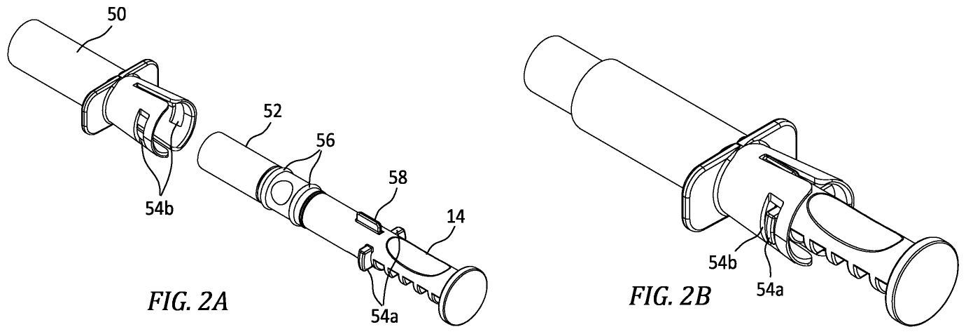

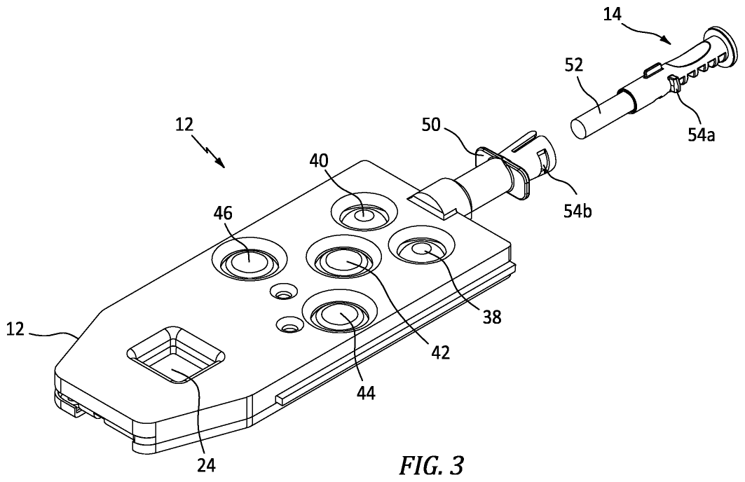

FIGS. 2 and 3 show the sample collection device 14 in greater detail. The sample collection device 14 is inserted into an inlet port in the form of a syringe barrel 50 of the body 12 in greater detail. The sample collection device 14 comprises an absorptive pad 52, locking tabs 54a, one or more seals 56, and a guide rail 58. The guide rail 58 ensures that the locking tabs 54a correctly align with locking slots 54b. The absorptive pad 52 is placed in the mouth of a person to collect a biological sample 60 by absorbing the saliva into the pad 52. When the sample collection device 14 is inserted into the syringe barrel 50, the seals 22 create a substantially airtight seal between the sample collection device 14 and the syringe barrel 50 with the absorptive pad 52 sealed inside the syringe barrel 50. Depressing the syringe into the sample port compresses the pad 52 and releases the sample 60 into the sample port. The sample 60 enters the body 12 through the sample port 36 for isolation, concentration, amplification, and detection of any targeted nucleic acids in the sample 60. As known to those of ordinary skill in the art, known amplification techniques exist to amplify DNA and RNA sequences, and thus the invention could be used for either. The invention contemplates other mechanisms to secure the sample collection device to the body, such as a sample collection device which screws into a syringe barrel and other mechanisms known to those of ordinary skill in the art.

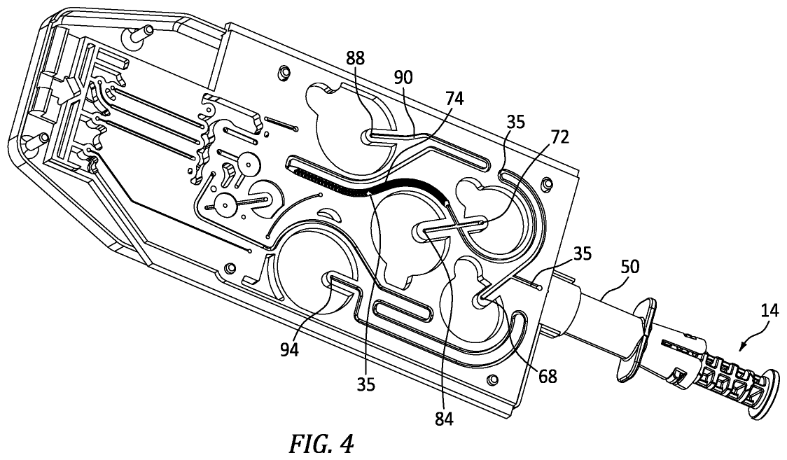

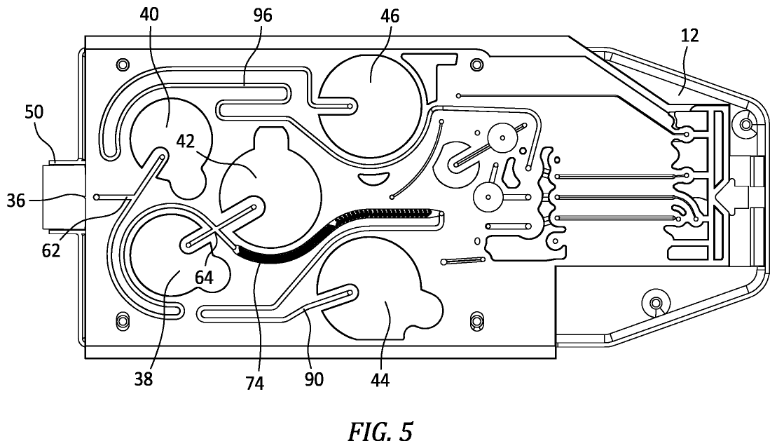

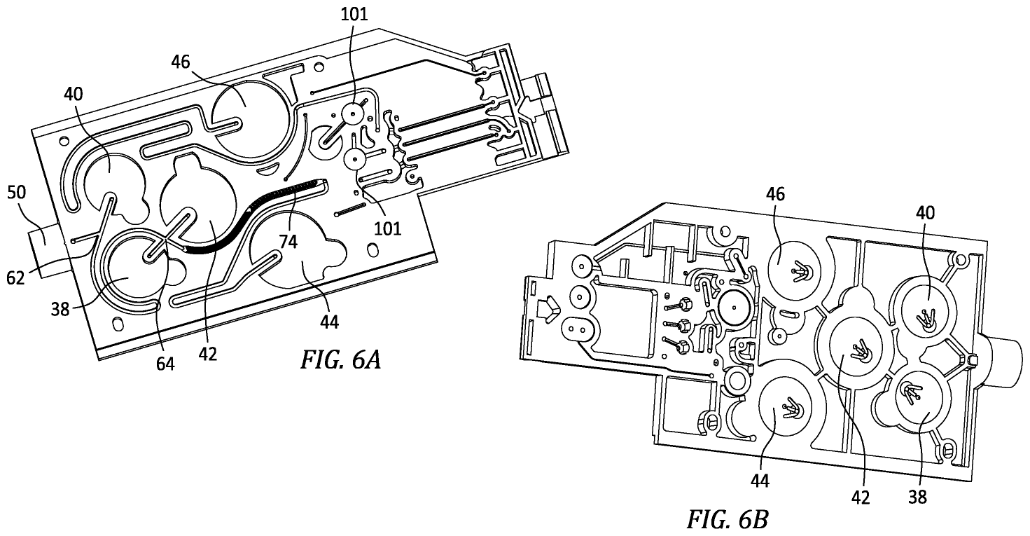

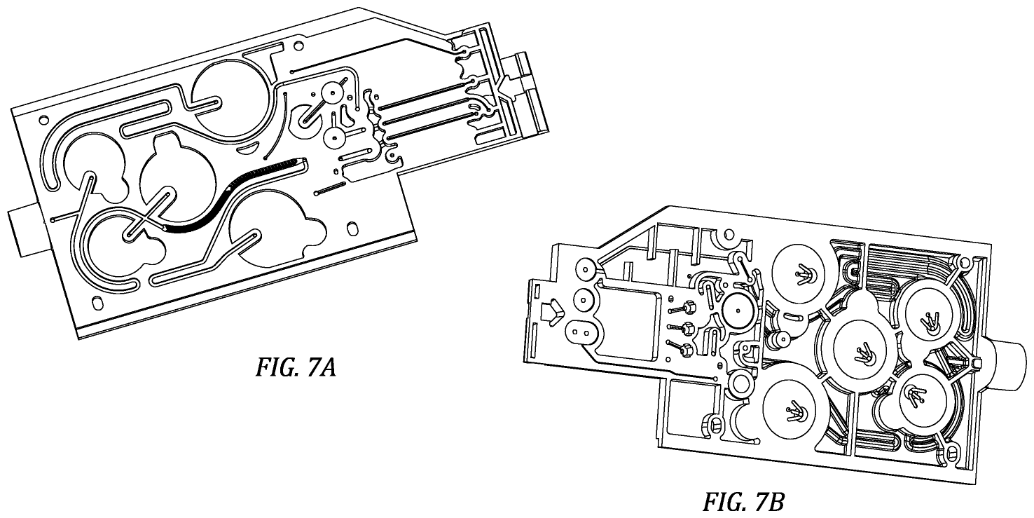

FIGS. 4-7 show an exemplary embodiment of the body 12 in greater detail, with FIGS. 4 and 5 showing the flow paths on the underside of the body 12 and FIGS. 6 and 7 showing both the top and bottom of the body 12. The syringe barrel 50 is partially shown. When the sample collection device 14 is locked in place, the absorptive pad 52 of the sample collection device 14 is compressed, releasing the sample 60. The released sample 60 is in fluid contact with the sample port 36 located inside the syringe barrel 50, allowing the sample 60 to flow into the sample conduit 35 of the body 12. The sample 60 flows into sample conduit 35 through capillary action and/or by the pressure created by inserting the sample collection device 14 into the syringe barrel 50. As indicated above, the five stations of the device include sealed containers or blisters 49, i.e., the lysis blister 38, the first wash blister 40, the second wash blister 42, the elution blister 44, and the negative control blister 46. Depression of the blisters 49 forces the fluids within the blisters 49 into the respective fluid channels of the device.

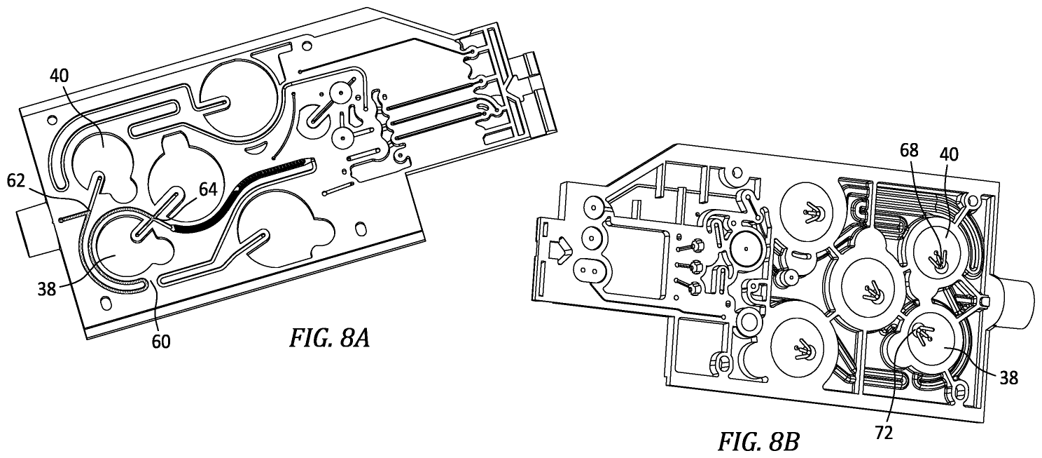

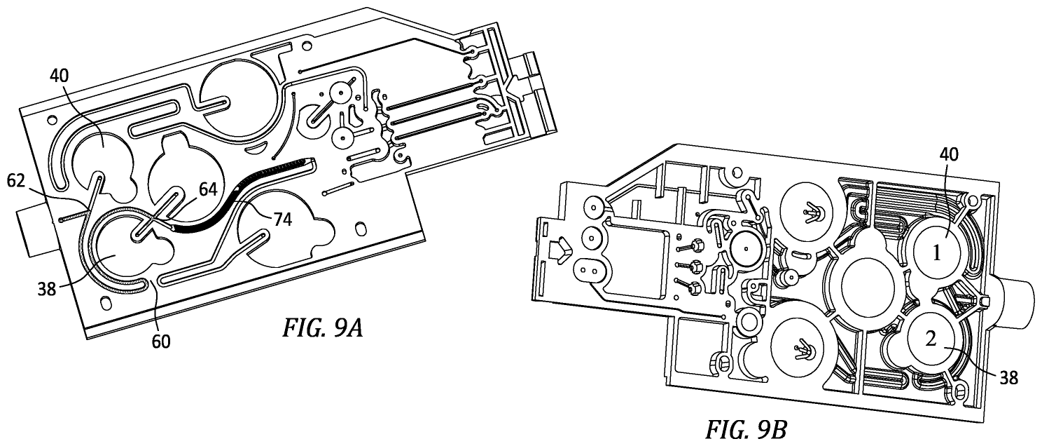

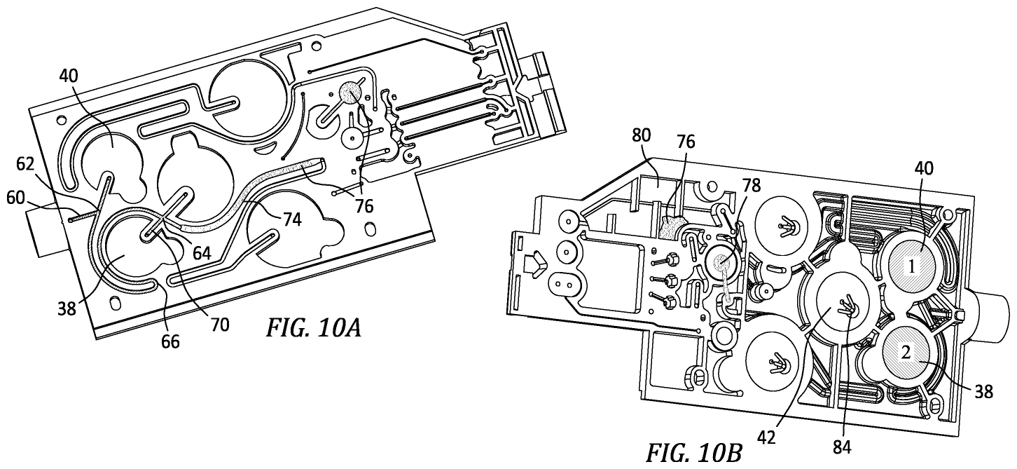

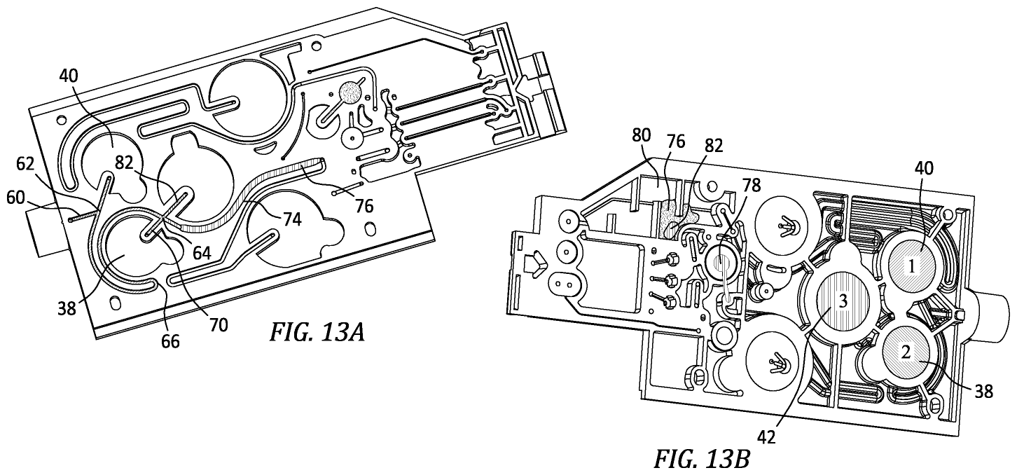

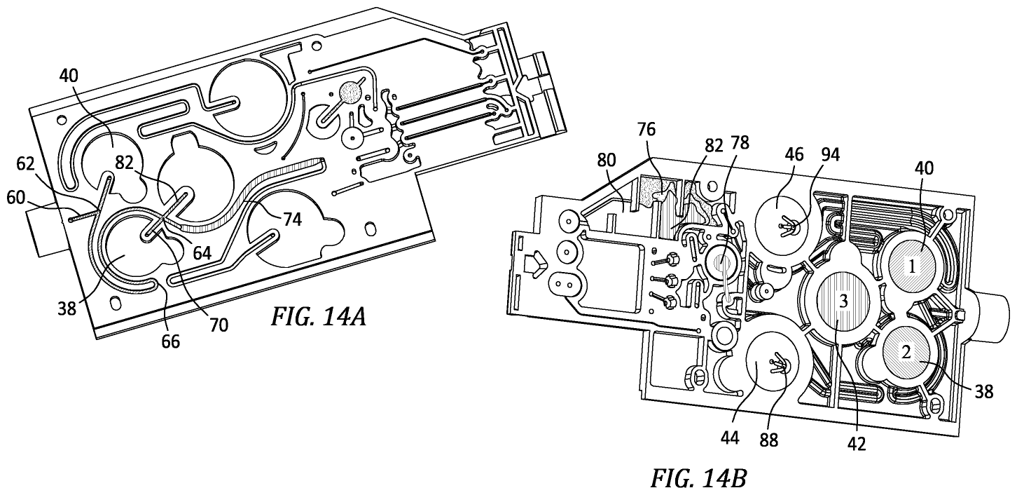

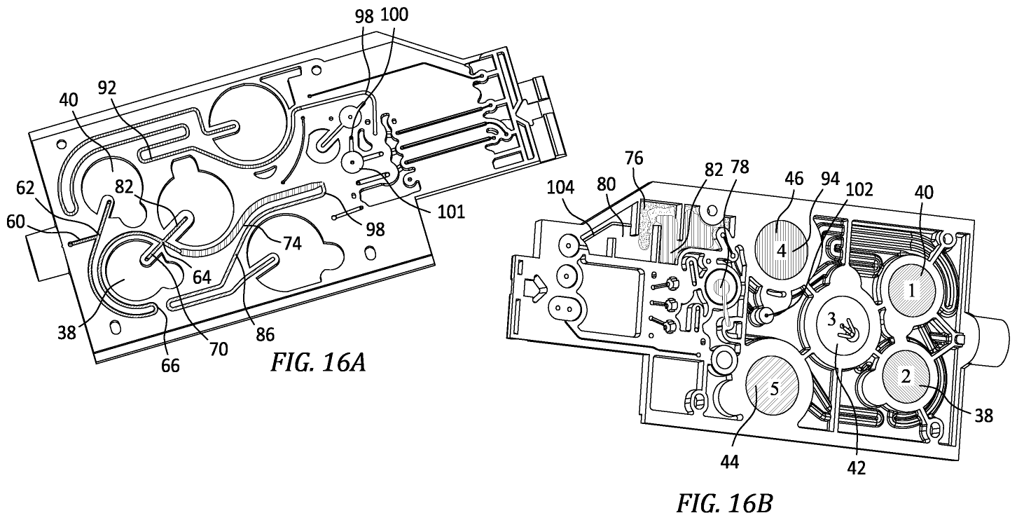

FIGS. 8-17 demonstrate the fluid flow paths associated with a method of using the device. First, as shown in FIGS. 8A and 8B, the sample 60 is delivered to the body 12 and proceeds sufficiently into the body 12, i.e. sufficiently ahead of a first wash buffer junction 62 to reach or almost reach a lysis buffer junction 64. Second, as shown in FIGS. 9-10, the first actuator or button 18 is depressed releasing both the first wash blister 40 and the lysis buffer blister 38 at approximately the same time. This releases the first wash buffer 66 through the first wash buffer inlet 68 progressing the sample 60 further down the sample conduit or channel 35 at the same time releasing the lysis buffer 70 through the lysis buffer inlet 72. In a microfluidic environment, the sample 60 and the lysis buffer 70 pass through the lysis buffer junction 64 and mix in the mixer fluid channel 74. As the lysis buffer 70 is mixed with the sample 60, the cell walls of the cells and cell nuclei in the sample 60 are disrupted in the resulting lysed sample solution 76, releasing the RNA or DNA within the cells into the solution. The continued entry of the first wash buffer 66 and lysis buffer 70 into the sample conduit or channel 35 causes the continued mixture of the lysis buffer 70 with the sample 60, while the resulting lysed sample solution 76 continues to flow through the channel 35, exiting the mixer fluid channel 74, passing through the membrane 78, and into the waste chamber 80. As the lysed sample solution 76 passes through the membrane 78, the RNA or DNA in the lysed sample solution 76 adsorbs to the membrane 78. More DNA or RNA is adsorbed if ethanol is present on the membrane 78, and accordingly the lysis buffer 70 and first wash buffer 66 may contain sufficient ethanol to improve adsorption. As the first wash fluid 66 subsequently passes through the membrane 78, matter and fluids in the lysed sample solution 76 (other than the DNA or RNA adsorbed or adhered to the membrane 78) are washed into the waste chamber 80, substantially isolating DNA and/or RNA on the membrane. It will be understood by those of ordinary skill that the washes are imperfect; some undesired matter may remain and some DNA or RNA may be washed away.

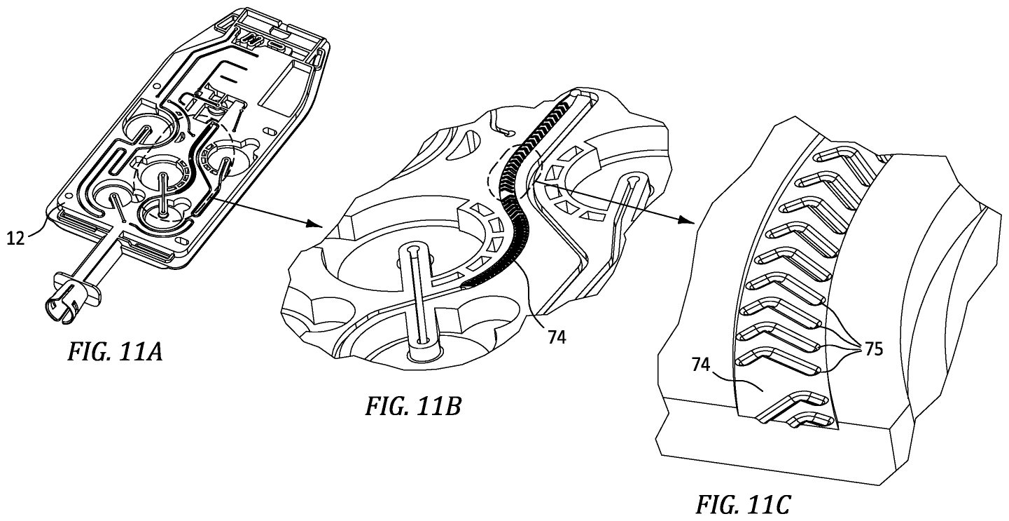

The mixer fluid channel 74 shown on the body 12 is a herringbone passive mixer; a magnified detail of a herringbone mixer is shown in FIGS. 11A and 11B. A herringbone mixer creates a substantially homogeneous mixture of two fluids under laminar flow, in this case to mix the test sample with the lysis buffer. When two fluids meet at a microfluidic junction, unless the flow rate is high, mixing will generally occur by only diffusion. In the device 10, mixing the sample 60 and lysis buffer 70 by diffusion will not generally be fast enough for the lysing process to complete before the sample-lysis mixture reaches the membrane; a passive mixer such as a herringbone mixer ensures a substantially complete mixing of the fluids as they travel downstream and an effective lysing process.

The herringbone mixer used herein is defined by a plurality of axially-spaced recesses or grooves 75 formed in the sample conduit 35, where the axially-spaced recesses or grooves 75 are formed in a bottom wall of the sample conduit 35. As can be seen, the recesses or grooves 75 are shaped such that a first part of each groove 75 imparts a first rotational component to a fluid flowing therethrough in a first direction, and a second part of each groove imparts a second rotational component to the fluid flowing therethrough in a second direction that is different than the first direction, to thereby facilitate mixing the sample 60 and lysis buffer 70 into the sample-lysis mixture. As also shown, the first part of each recess or groove is oriented approximately at an acute angle relative to the second part of each recess or groove, and the first part of each recess or groove is shorter than then second part of each recess or groove. The plurality of axially-spaced recesses or grooves 75 define an approximate herringbone shape. In the illustrated embodiment, the first part is about ⅓ the overall length of the respective groove and the second part is about ⅔ the overall length of the respective groove. Also in the illustrated embodiment, approximately equal amounts of the saliva sample and lysis buffer are introduced into the sample conduit, and the passive mixer creates a substantially homogenous mixture of these components in an approximately 1:1 ratio. However, as may be recognized by those of ordinary skill in the pertinent art based on the teachings herein, other relative amounts of components may be mixed as desired or otherwise required.

To ensure the sample 60 is adequately lysed, i.e. that cell walls within the sample 60 are sufficiently disrupted to ensure that a sufficient amount of nucleic acids therein are released into solution, it is important to ensure that a sufficient quantity of the lysing agent within the lysis buffer 70 mixes with the sample 60. Accordingly, in a microfluidic environment, the flow rate of the sample 60 relative to the flow rate of the lysis buffer 70 is preferably controlled. In the illustrated embodiment, the flow rate of the lysis buffer 70 is determined primarily by the rate at which the lysis blister 38 empties and the flow rate of the sample 60 is determined primarily by the rate at which the first wash blister 40 empties. Accordingly, the rate at which the lysis blister 38 empties relative to the rate at which the first wash blister 40 empties are preferably within a desired or predetermined range. The present inventors have found that with uniform source volumes for the two blisters 38, 40, and approximately equal pressures applied to the two blisters 38, 40, altering the length of the microfluidic conduits provides sufficiently precise control of the liquids entering the passive mixer to ensure the intended fluids are adequately mixed. In the illustrated embodiment, the first wash blister 40 and lysis blister 38 are emptied at approximately equal rates by applying approximately equal pressures to the blisters. As described below, such equal pressures can be applied by using dual plungers driven substantially simultaneously by a common drive spring or springs into the blisters.

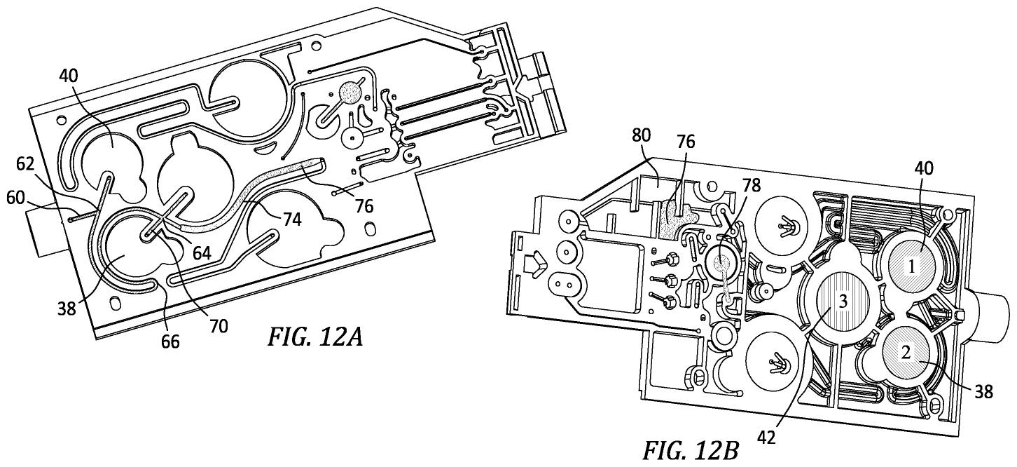

Next, as shown in FIGS. 12-14, the second button 20 is pressed, pushing on the second wash blister 42, which releases the second wash buffer 82 into the body 12 through the second wash fluid inlet 84. The second wash 82 fluid entering the channel advances any remaining lysed sample solution 76 present in the channel through the membrane 78 and into a waste chamber 80. The second wash buffer 82 continues to wash the membrane 78 in the manner described above, and the second wash fluid may or may not be identical to the first wash fluid.

Finally, the third button 22 is depressed, in turn depressing the elution buffer blister 44 and the negative control blister 46. As shown in FIGS. 15A and 15B, the elution buffer 86 begins to enter through the elution buffer inlet 88 and into the elution buffer channel 90 at the same time as the control buffer 92 begins to enter through the control buffer inlet 94 and enter the control buffer channel 96. The elution buffer channel 90 and the control buffer channel 96 are both elongated and initially filled with air. The control buffer channel 96 is substantially longer than the elution buffer channel 90. The air 98 in the elution buffer channel 90 proceeds ahead of the elution buffer 86 through the membrane 78, pushing the remaining wash fluid 82 into the waste chamber 80. When the elution buffer blister 44 and negative control blister 46 are partially depressed, the air 98 passes through the membrane 78, thereby drying it. After the membrane 78 is sufficiently dry, the waste chamber vent 102 on the waste chamber 80 is closed and the vents on the reaction chambers 24 are opened. The length of the elution buffer channel 90, and accordingly the volume of air 98 in the channel, is specifically calculated to ensure that the volume of air 98 passing over the membrane 78 is sufficient to dry the membrane 78 to at least the degree necessary to prevent ethanol poisoning from disrupting the test.

The waste chamber vent 102 is connected to the waste chamber 80 through a tortuous waste chamber vent conduit 104 from the far end of the waste chamber 80 to a location approximately between the elution buffer blister 44 and the negative control blister 46. This elongated path places the waste chamber vent 102 in a location where it can be closed by pressing the third button 22. In the microfluidic environment of the device, closing the waste chamber vent 102 substantially prevents the entry of further fluids into the waste chamber 80. Opening the reaction chamber vent allows fluids to enter the reaction chamber conduit 100 while at about the same time sealing the waste chamber 80, directing fluids into the reaction chambers 24.

A hydrophobic valve sits at the beginning or middle of the waste chamber vent conduit preventing liquids in the waste chamber from exiting the device. Because the liquids in the chamber can be hazardous chemicals or biological waste, preventing escape of the fluids is important. Liquids may not pass the hydrophobic valve, but some aerosolized particles may. Because the gas inside the chassis is vented and is likely saturated with water and other volatiles at a rate related to the vapor pressure of the water or other volatiles and the temperature of the surface from which the evaporation is to occur, and because the air at the exit of the vent tube is at the local ambient composition, there is a changing vapor concentration gradient within the device leading from the vent to the outside air. All gas from that ambient location back to the evaporation surface has a variable and increasing level of vapor comprising water and other volatiles. The time to equilibrium is equal to the tube length squared divided by the diffusivity of the molecule that is evaporating. If the length of the tube is great enough and diffusivity low enough, the evaporating molecules will not exit the device until the measurement is over. Based on the diffusivity of water vapor in air of 0.24 cm²/second, if the duration of a test is 20 minutes, the leading edge of the diffusing vapor will have progressed a distance of about 17 cm. If the tube length is only 10 cm, the time for the diffusing molecules to reach the exit is about 7 minutes. In any test with diffusing molecules which should not escape, or in which interference by outside air is problematic, the length of the vent tube may be calibrated appropriately to ensure that volatiles do not escape before the test completes and that the outside air does not interfere with the test.

For this reason, long vent tubes with small cross-sectional areas are added to the distal side of the vent, leading to an eventual opening to the environment or test room conditions. Any water molecules that permeate the vent membrane transportation will be slowed by diffusion that is limited by the small cross section of the tube. A saturation gradient will be developed between the source of water molecules, the vent membrane distal end, and the exit to the atmosphere. This diffusion gradient will slow evaporation and limit the entry of air into the reaction chamber. Because the mass flow is very low, the cross-sectional area of the tube can be extremely small without creating a viscous pressure drop or other adverse flow consequences; the practical limitation on tube cross-sectional area is set by fabrication capability. Preferably, it is made as narrow as practicable given the technologies used to make the chassis. For instance, a passage that is preferably within the range of about 75 um to about 125 um wide by about 15 to about 35 um tall, and more preferably about 100 um wide by about 25 um tall, would suffice and is within the ability of known manufacturing techniques.

Initially, before the chassis is filled with liquid, the gas in the chassis is simply air. The vent tube gas remains as “pure” air up until the moment the fluid within the chassis is added. For a period of time from adding the fluid until there is a steady vapor concentration gradient within the device, the concentration of evaporating molecules is changing. This period of time can be estimated based on the following formula:

Time to equilibrium is equal to the tube length squared divided by the diffusivity of the molecule that is evaporating.

If the length of the tube is great enough and diffusivity low enough, the evaporating molecules will not exit the device until the measurement is over.

In at least some embodiments, the reaction chamber vents are closed by covering the chambers with a plastic film or foil. Because the plastic film traps the air, the vent is closed. In such environments the reaction chamber vents may be opened by piercing the film with, for example, a snake tooth shaped piercing agent. In the embodiment shown, the waste chamber vent located between the elution buffer blister and the control blister terminates a waste chamber vent path. In some such embodiments, the reaction chamber vents may be connected to a reaction chamber vent path terminating close to the waste chamber vent. In this way, a single button press could rupture the blisters, close the waste chamber, and open the reaction chamber vent. In other embodiments, the reaction chamber vent is never sealed; instead a Laplace valve is used to direct flow. The Laplace valve will direct fluids first into the waste chamber and then into the reaction chambers. The Laplace valve connected to reaction chamber conduit 100 will be opened by closing the waste chamber vent; by blocking flow to the reaction chamber the pressure in the valve will increase such that fluids will flow down the reaction chamber conduit.

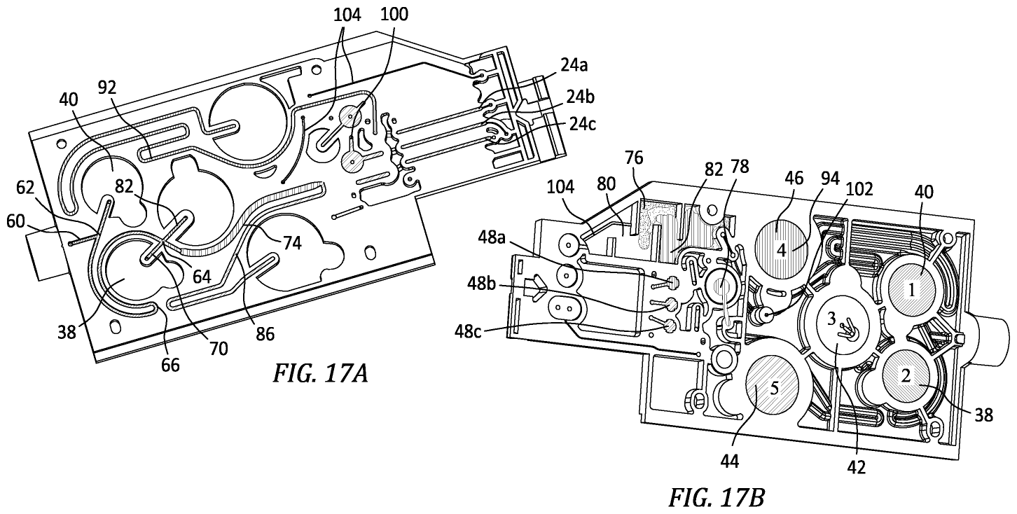

The elution buffer 86 will remove adsorbed DNA or RNA from the membrane 78; such DNA or RNA will enter the elution buffer 86 and proceed into the reaction chamber conduit 100 for amplification and detection. The reaction chamber conduit 100 contains reconstitution chambers 48b, 48c and reaction chambers 24b, 24c. The reconstitution chambers 48 contain dried reagents needed to complete the amplification reaction, and are arranged in a manner such that the reagents will be dissolved into the elution buffer 86 with any eluted DNA or RNA therein. In at least some embodiments, the dried reagents take the form of lyophilized beads. The reagents also may be “warm start” reagents, which are optimized for use in a warm environment. When using “warm start” reagents, the reaction chambers are preferably preheated prior to introducing the reaction fluids therein, as described further below. In FIGS. 17A and 17B three reconstitution chambers are shown: one for the negative control, one for the positive control, and one for the test; those of ordinary skill could eliminate the reconstitution chambers, or alter the number of reconstitution chambers as needed, such as to ensure that adequate reagents are available for the amplification reaction. The amplification reaction will occur in the reaction chambers 24. A series of Laplace valves 101 are located in fluid communication between the reconstitution chambers and the solid-state membrane to control the flow and/or the order of flow from the solid-state membrane into the reconstitution chambers and associated reaction chambers. In one embodiment, the Laplace valves 101 are configured to initially direct the eluent and captured nucleic acids into a first reconstitution chamber and associated test reaction chamber, and then direct such flow into a second reconstitution chamber and associated internal control chamber. In some embodiments, a Laplace valve also is in fluid communication with the waste chamber and is configured to first fill the waste chamber before the reconstitution and reaction chambers, as described above. In an alternate embodiment, a Laplace valve will be downstream of the reconstitution chambers, directing fluids first into the test reaction chamber and then into the internal control chamber. In the illustrated embodiment, the fluids which enter the test chamber(s) are the first elution fluids to pass through the membrane, which the inventors have found may contain the most DNA or RNA or be more nucleic acid rich than the subsequent fluid flow. Finally, some embodiments may use a Laplace valve with initially sealed reaction chamber vents to control the flow of contaminating air into the reaction chamber and to control the flow of potentially hazardous air out of the device.

As shown in FIGS. 15-17, the negative control channel 96 is elongated, taking an indirect path to the negative control reconstitution chamber 48a and reaction chamber 24a. The length of the negative control channel 92 is calculated to ensure that the negative control blister 46 is able to depress to approximately the same degree as the elution buffer blister 44, and accordingly the total of (i) the negative control channel volume, (ii) the negative control reconstitution chamber volume, and (iii) the negative control reaction chamber volume must be approximately equal to the total of the volume of (i) the elution buffer channel, (ii) the test reconstitution chambers, (iii) the test reaction chamber, and (iv) the internal control chamber. In this way, blisters of a uniform size will generate predictable results. The inventors have found this setup is relatively simple to manufacture while ensuring adequate emptying of the blisters. As will be discussed later in greater detail, the button configuration contemplated with this invention uses a horizontal bar or plunger mount with dual plungers and an associated plunger spring to substantially simultaneously drive the plungers into the blisters and substantially simultaneously dispense the liquids therefrom. The bar or plunger mount has a limited ability to bend, and thus it is important that both blisters are able to empty adequately, or one blister may not be able to empty fully. Of course, one of ordinary skill will understand based on the teachings herein that there are alternate mechanisms for ensuring that all blisters empty adequately, including overflow-style Laplace valves which allow the blister to continue to empty after fluids have flowed completely through the desired paths. In addition, one will understand based on the teachings herein that alternative button configurations, such as those with a horizontal bar which can rotate, will effectively transfer pressure to the blisters. In some situations, however, simultaneous emptying of blisters may be important, such as the first button, where the simultaneous flow of the first wash buffer pushing the sample and the lysis buffer may be necessary to ensure that the sample moves into the passive mixer with the lysis buffer; if the first wash buffer was not pushing the sample forward, the lysis buffer would enter the mixer alone.

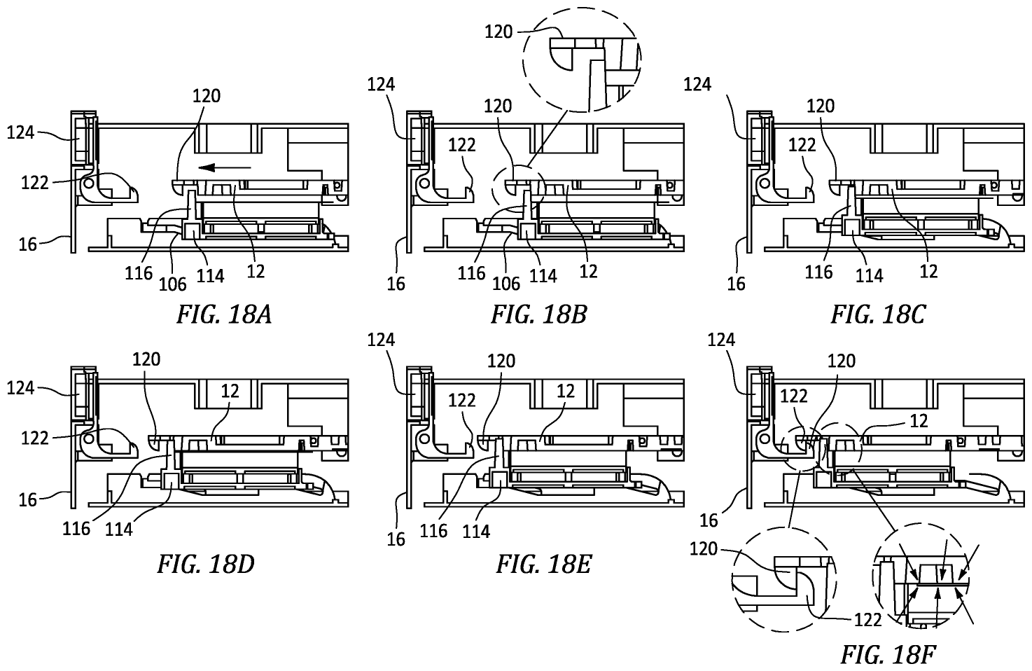

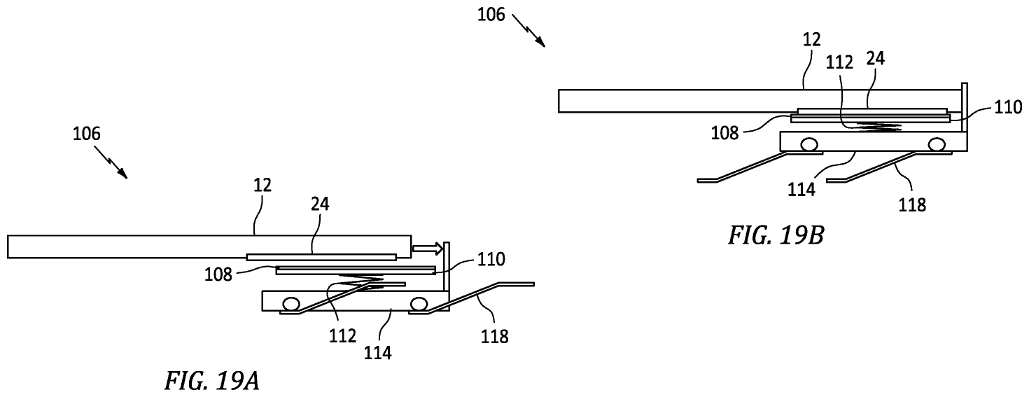

In FIGS. 18-20, a heater assembly 106 of the base station 16 is shown. The heater assembly 106 is used to provide the heat required for the amplification reaction, which may require keeping the reaction chambers at approximately 60° C. for a period of time. A sled-and-ramp system is used to move the heating element 108 of external heater assembly 106 into contact with the bottom of the body 12 upon insertion of the body 12 into the base station 16. The external heater assembly 106 shown comprises a heating element 108 connected to a plate 110, which is pushed upwards by a spring 112 connected to a sled 114. At one side of the sled 114 is a sled tang 116; when the device body contacts the sled tang, it moves the sled 114 up a ramp 118 until the spring 112 compresses and the heating element 108 is firmly in contact with an area of the body 12 directly under the reaction chambers 24. In some embodiments, a latch 120 on the device body 12 engages a latch 122 either on the external heater assembly 106 or, as shown, on the base station 16. A button 124 may be present to disconnect the latches 120, 122. The button 124 shown is connected to the latch 122 on the base station 16, by pressing the button the base station latch 122 is lowered, disconnecting it from the latch 120 on the device body 12. The spring 112 pushes the plate 110 to provide a desired contact pressure between the heating element 108 and the device body 12 and to allow for dimensional tolerances of the sled-ramp components, thereby placing the heating element 108 in firm contact with the bottom of the body 12. The heating element 108 may be activated prior to filling the reaction chambers 24 in order to preheat them, such as upon insertion of the body 12 into the base station 16. For example, the heater 108 may be activated automatically by the flexible circuit board 126 upon movement of the body 12 and/or heater into the operational position, or may be activated upon movement of one of the buttons 18, 20 or 22 from its non-actuated position to its actuated position. Preheating the reaction chambers 24 decreases the time required for the reaction to complete, in some cases by at least as much time as it takes to insert the body 12 into the base station 16 and press each of the three buttons 18, 20, 22, which may be at least about 30 seconds or more. Further, when “warm start” reagents are used, preheating the reaction chambers 24 creates conditions optimal for the reaction. After the heating phase of the reaction is complete, the body 12 is released and removed from the base station, and the external heater assembly 106 and sled are moved downwardly and out of engagement therewith. The sled-and-ramp system allows for actuation without separate, additional actions from the user beyond inserting the device body 12 into the base station. In addition, the external heater assembly 106 is moved into contact with the device body 12 while prohibiting relative motion between the heater element 108 and device body 12 in the horizontal plane to thereby prevent wear on the heater element 108. As may be recognized by those of ordinary skill in the pertinent art based on the teachings herein, the sled-and-ramp system can move other elements into place as well.

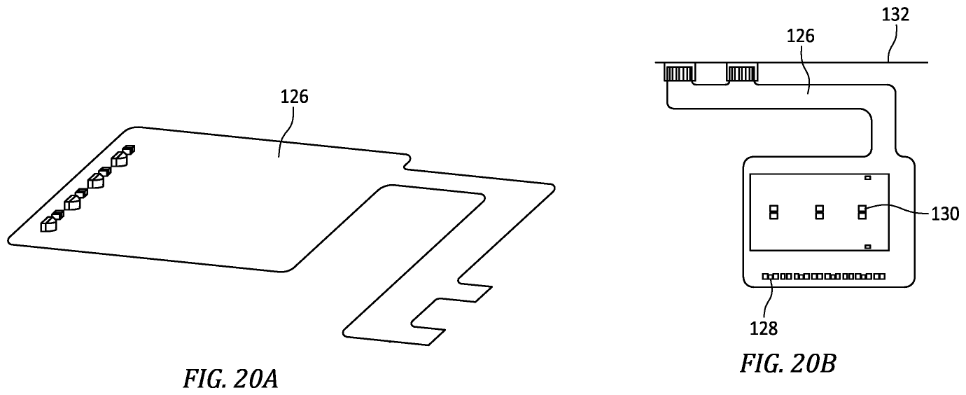

A flexible circuit board 126 shown in FIG. 20 mounts light emitting diodes (LEDs) 128 that are used to illuminate and highlight the reaction chambers and sensors 130 for the heater 106. FIG. 20B shows the position of the LEDs 128 and the sensors 130. The sensors 130 will be underneath the reaction chambers 24 and approximately aligned with the heating element 108, whereas the LEDs 128 will be to the side of the reaction chambers 24. As shown in FIG. 20A, the LEDs 128 are positioned on top of the flexible circuit board 126 so that they can shine light parallel to the surface of the heater 108.

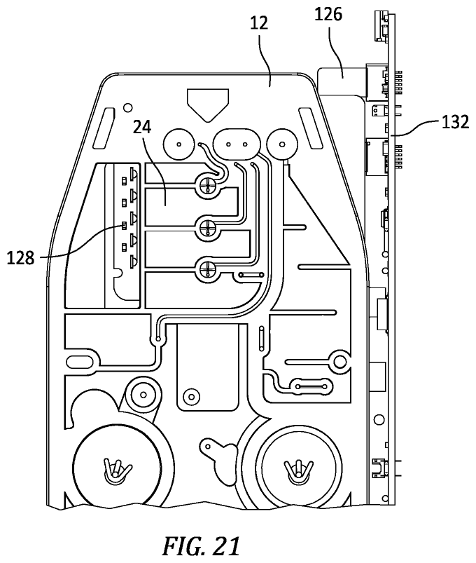

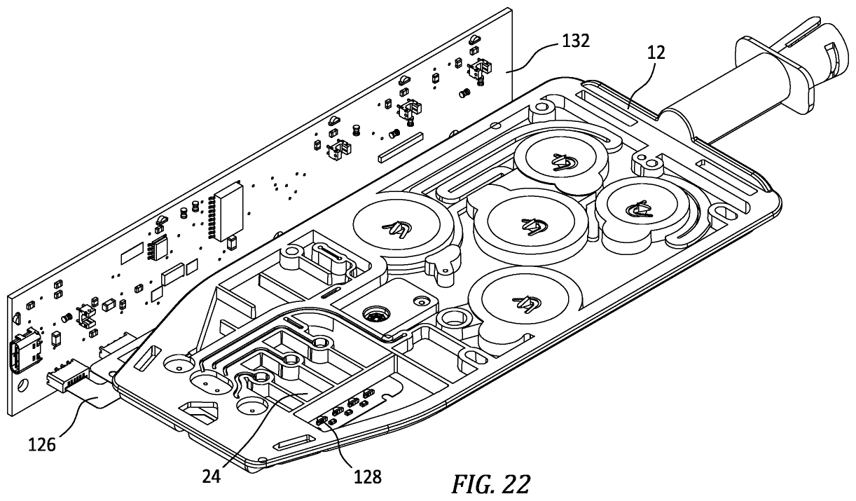

FIGS. 21 and 22 show the positioning of the LEDs 128 in more detail. The LEDs 128 are alongside a vertical surface of the body 12, and in line with the reaction chambers 24 of the body 12, and the light generated by the LEDs 128 will shine in the direction of the body 12. Because at least a portion of the body 12 around the reaction chamber 24 is at least partially transparent, and preferably the whole chassis is transparent or substantially transparent, the light will shine through to the reaction chambers 24. The light will be an activating or stimulating light. Target fluorescent chemical compounds are present in any of the reaction chambers 24 and such reaction chambers will fluoresce in response to the activating or stimulating light transmitted therein. In some embodiments, fluorescent chemicals are produced by the LAMP reaction; in others the fluorescent chemicals are initially present and destroyed by the LAMP reaction. In either case, a LAMP reaction will only occur in the presence of the target sample, and thus if the fluorescence of the test reaction chamber 24c matches the fluorescence of the positive control reaction chamber 24b, a positive result is indicated, and if the fluorescence of the test reaction chamber 24c matches the fluorescence of the negative control reaction chamber 24a, a negative result is indicated. In a COVID-19 test, Calcein may be used as the indicating chemical. After a successful LAMP reaction, Calcein in solution will attach to the amplified nucleic acids and respond to an excitation light by fluorescing. Accordingly, if the test reaction chamber 24c shines in fluorescing light, this indicates a positive result, whereas if the test reaction chamber 24c remains dark, this indicates a negative result. In at least some embodiments, the reaction will take approximately 20 minutes to complete and, accordingly, after about 20 minutes the reaction-complete indicator 34 will illuminate, change color or otherwise change state to identify that the reaction is complete. A sample 60 which contains a high concentration of target nucleic acids may show a positive result in less than 20 minutes, in some cases a positive result may be indicated in about 5 minutes or less. As indicated above, preheating the reaction chambers can contribute significantly to reducing reaction times. The results may be viewed through the reaction chamber window 26, as shown in FIG. 1.

FIGS. 21 and 22 also show a printed wiring board 132. The printed wiring board 132 is connected to a power source (not shown), the stimulating LEDs 128, the heating element 108, the lights indicating the stage of operation including the first button indicator 28, the second button indicator 30, and third button indicator 32, and the reaction complete indicator 34. The board 132, which may include a number of logical units, transistors, capacitors, and other passive and active electronic components, ensures that the various steps of operating the device are indicated to be performed, in the correct order. The board also controls the temperature of the LAMP reaction by modulating power supply to the heating element 108 in response to information from the heat sensors 130. In some embodiments, such as embodiments intended for the general public to use with at-home tests, logic and indicators may be present to indicate that the device has not been used properly, as well as a speaker and logic to give audible queues indicating how to operate the device. The audible queues may include words in the language of an expected user such as “press button 1 now” or “reaction complete.”

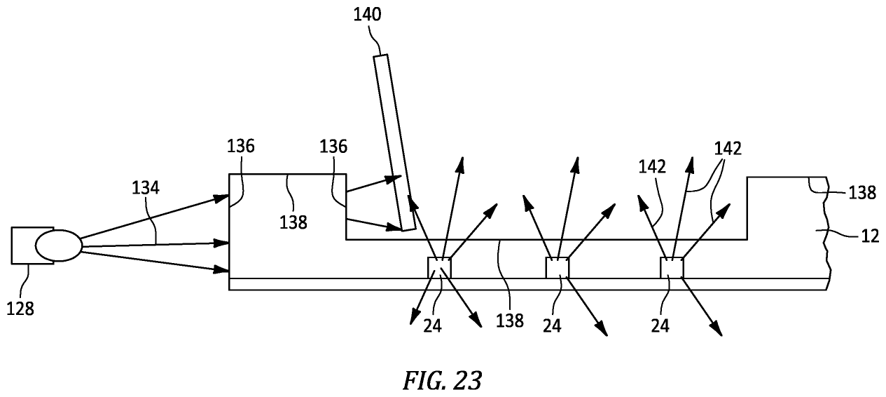

In FIG. 23, a demonstration device shows how light passes through the device. Light will be used to aid visual detection of DNA or RNA amplification. The demonstration device could be used with either of two primary means of making visual detection of DNA or RNA amplification. First, the sample's spectral absorption could change during a LAMP reaction, which will be observed by changes in the absorption of light, either broad spectrum or selected colors. The illumination light is filtered by the medium and then directed to the observer, who will see a different color in sample with amplified DNA or RNA than in a sample without amplified DNA or RNA. Positive and negative control channels show the color of a positive and negative result, and indicate a successful reaction: if the color of the positive and negative channels matches, the reaction did not proceed successfully. Alternatively, visual detection may use fluorescence. In this case, the sample is exposed to light that stimulates the fluorescence and the observer selectively observes the fluorescence (ignoring or being shielded from the stimulating light). If the wavelength of the stimulating light is in the ultraviolet range, which the human eye cannot see, the stimulating light will not be a distractor. However, some stimulating lights are a similar wavelength to the wavelength of the fluorescent light, and an alternate means of filtering out the stimulating light is needed. In order to avoid expensive passband filters, at least some embodiments of the device or method make use of the fact that the stimulating light can be directional and that the fluorescing light is omni-directional. The light transmission and guiding nature of the transparent chassis material is used to direct light throughout the chassis and especially through the walls and top of the reaction chambers. This directed light mostly stays within the structure and only escapes readily through incidence at near normal angles to a surface. As a result, the observer who views the top of the reaction chambers sees very little of the stimulating light which is being directed laterally through the structure in this area. The stimulating light passes readily into the reaction chambers by virtue of the matching of index of refraction of the chassis material and of the fluid. The stimulating light in the reaction chamber generates the fluorescing light which is emitted in all directions. That fluorescing light thus has a significant fraction of its light that escapes in the direction of the observer. This process greatly enhances the observed ratio of fluorescing light to stimulating light.

The device shown in FIG. 23 embodies these principles. One or more LEDs 128 shine light into the device body 12 in a direction 134 parallel to the bottom of the device. Surfaces 136 normal to the direction of the light 134 allow the light to pass through easily, whereas light is generally contained by surfaces 138 roughly parallel to the direction of the light 134. In some cases, a shield 140 may be used to block light from exiting the device in undesired ways. In contrast to the stimulating light, the fluorescing light shines in all directions 142, including towards the observation window. The fluids in the reaction chambers 24 react differently to the light depending on whether a positive test result or a negative test result is indicated.

The body 12 may be substantially transparent throughout, and at least a portion of the body 12 including the reaction chamber 24 is substantially transparent, and includes a substantially transparent top surface 138 extending over the reaction chambers 24 and two substantially transparent side surfaces 136 extending downwardly from the top surface 138 along opposite sides of the reaction chambers 24 relative to each other. Each reaction chamber 24 is visually observable in a viewing direction through the substantially transparent top surface 138, and the device further comprises a stimulating light source 128 located adjacent to a substantially transparent side surface 136 configured to transmit stimulating light through the side surface 136 and reaction chambers 24 in a direction 134 substantially lateral or substantially perpendicular to the viewing direction. In some embodiments, the index of refraction of the substantially transparent body 12 and the index of refraction of the fluid in the reaction chambers 24 are configured to facilitate the passage of the stimulating light from the body 12 into the reaction chambers 24 to generate fluorescing light in the reaction chambers 24. The fluorescing light is emitted in substantially all directions 142 and is observable in the viewing direction through the top surface 138 of the body. As indicated above, preferably there is an observable difference to the human eye between the stimulating light and the fluorescing light to facilitate the ability of an observer to view the fluorescing light and distinguish it from any observed stimulating light.

The top surfaces of the body and the side surfaces of the body are smooth or polished to facilitate maintaining the stimulating light within the substantially transparent body. Ideally, the surfaces are optically smooth with respect to the wavelength and angle(s) of the stimulating light. No surface is absolutely smooth, if for no other reason than matter is composed of molecules in motion. An approximate criterion for smoothness is the Rayleigh criterion. A surface is reckoned to be optically smooth if d<λ/(8 cos θ), where d is the surface roughness (e.g., root-mean-square roughness height measured from a reference plane), λ is the wavelength of the incident illumination, and θ is the angle of incidence of this illumination. Thus, a surface that is smooth at some wavelengths is rough at others, or that is rough at some angles of incidence is smooth at others (e.g., near-grazing angles).

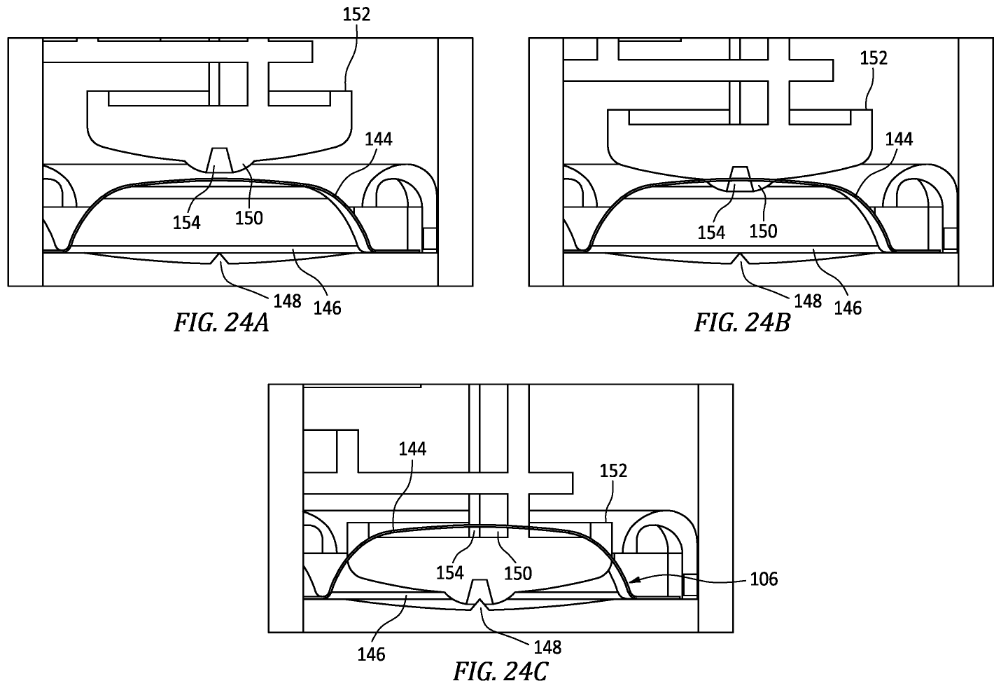

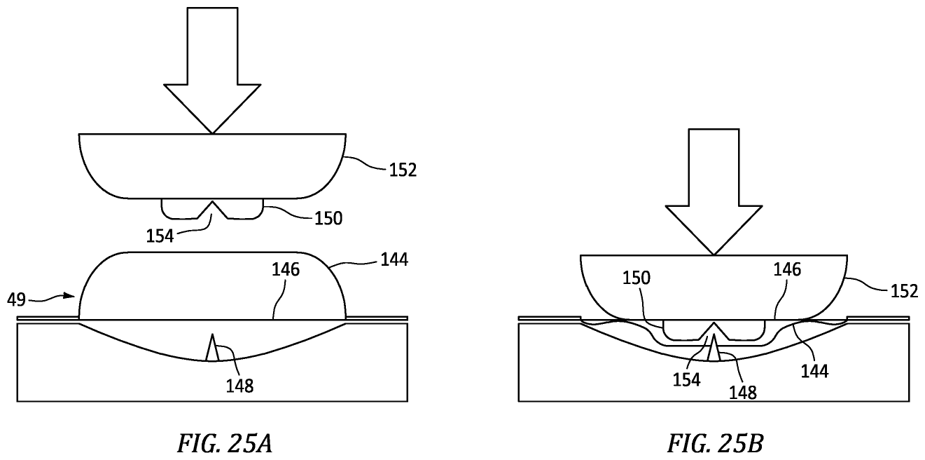

FIGS. 24-30 show the sealed containers or blisters 49 in greater detail. The sealed containers 49 may be blisters containing lysis buffers containing lysing agents, elution buffers containing elutes, stabilization solutions, wash solutions, or other mixtures as needed for specific tests. The blisters 49 are configured so that when the blisters 49 are compressed by applying pressure to each blister top 144, the frangible or breakable wall at the blister bottom 146 will rupture. In the illustrated embodiment of the blisters, a rupture pin 148 below the blister will pierce the blister bottom 146 when sufficient pressure is applied to the blister top 144, decreasing the required rupture pressure.

In at least some embodiments, such as those shown in FIGS. 24, 25, 28, and 29, the blisters 49 are compressed by manually-engageable actuators or plungers 152 which include the first and second chamber-engaging surfaces, where the second chamber-engaging surface or dome protrusion 150 defines a smaller width or diameter than the first chamber-engaging surface. This “bump on a bump” configuration facilitates in reducing the force required to rupture the blister due to the reduced diameter of the second chamber-engaging surface or dome-shaped protrusion 150. In other words, due to its lesser chamber-engaging surface area, the dome protrusion 150 reduces the manual force required to depress the blisters 49 and rupture the films or other frangible or breakable walls of the blister bottom 146 in order to release the liquids therefrom. After rupture, the first chamber-engaging surface or larger dome of the plunger 152 compresses the total diameter/shape of the blister top 144 to expel the remaining liquid therefrom. The dome protrusion 150 may contain a relief area or recess 154. The relief or recess 154 aligns with the rupture pin 148 such that when the plunger 152 is fully actuated, the rupture pin will be inside the relief area 154. This will prevent damage to the rupture pin 148 from the plunger 150, prevent damage to the plunger 150 from the rupture pin 148, and prevent the rupture pin 148 from piercing the blister top 144.

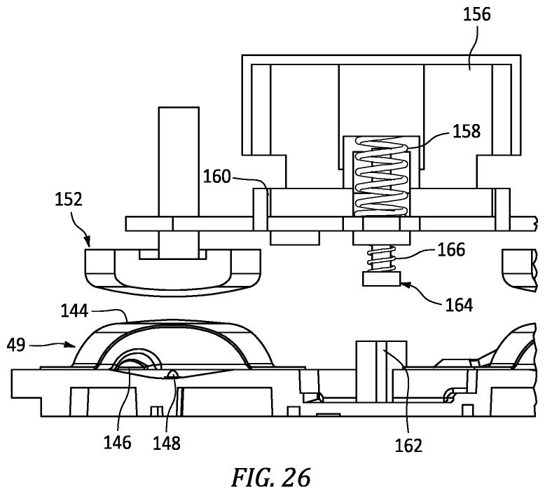

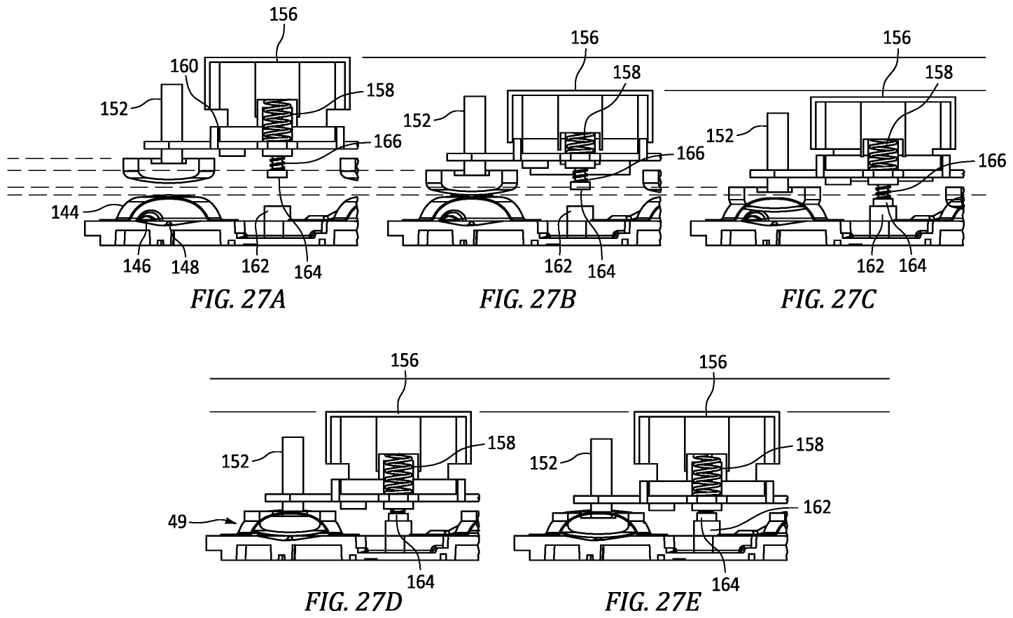

In FIGS. 26-29, a device to rupture the blisters 49 is shown which facilitates the substantially simultaneous rupture of two or more blisters 49. The device comprises a manually-engageable actuator or button 156, a plunger mount 160 mounted to the button 156, a plunger spring 158 defining a plunger spring force and mounted between the button 156 and the plunger mount 160, and a latch 180 of the button 156. Here, as shown in FIGS. 27A-27C, moving the button 156 from a non-actuated position to an actuated position, i.e. depressing the button 156, causes the plunger mount 160 to move from a first plunger position where the plungers 152 do not contact the blisters thereunder (FIG. 27A) to a second plunger position where the plungers 152 are in contact with the tops 144 of the blisters 49 (FIGS. 27B and 27C). Because the distance from the non-actuated position to the actuated position of the button is less than the distance from the first plunger position to the second plunger position, the plunger spring 158 is compressed by the button 152 from the top and by the resistance offered by the blisters 49 transferred to the spring 158 by the plunger mount 160. The plunger spring force or a portion thereof is sufficient to break the frangible or breakable wall of the blister bottom 146, releasing the fluids therein into the fluid channels of the device. As indicated in FIGS. 27D and 27E, the plunger mount 160 continues to push the two plungers 152 down under the force of the plunger spring 158, gradually emptying the blisters 49. Therefore, moving the button 156 from the non-actuated position to the actuated position causes the plungers 152 to rupture the blister bottoms 146, gradually releasing the fluids therein, and continues to compress the blisters 49 until they are substantially empty. One advantage of the spring-driven plungers is that they apply a user independent, and substantially uniform force to the blisters to dispense the liquids therefrom.

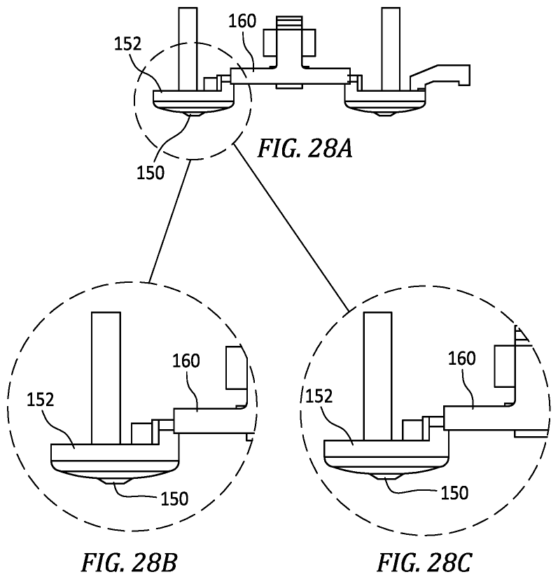

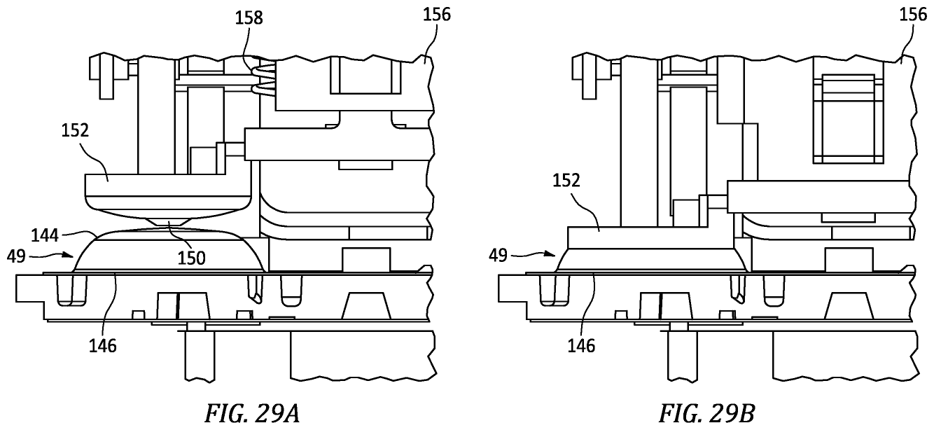

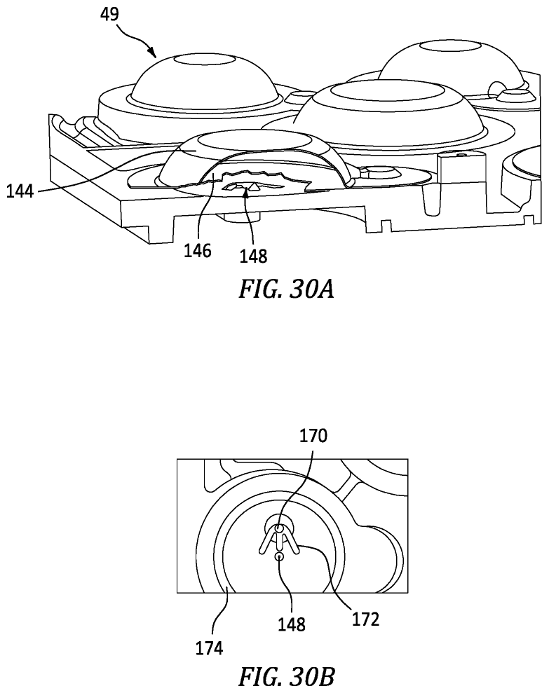

As shown typically in FIG. 28A, the plungers 152 may be spaced substantially symmetrically about the center of the bar or plunger mount 160. The plunger operation in rupturing and emptying the blisters is substantially the same whether one or multiple blisters are depressed. Smaller surface dome protrusions 150 may be formed on the bottom of the two or more plungers 152, as shown typically in FIGS. 28A-28C. As those shown in FIGS. 29 and 30, the button 156 locks into place after it is depressed, ensuring that the spring 158 continues to exert a downward force on the horizontal bar until both blisters are empty. As will be appreciated by those of ordinary skill in the relevant art based on the teachings herein, the plunger mount or bar may be designed to bow or otherwise flex when the button is depressed, which may negate the need for a spring. In addition, more than one spring may be used, and spring placement may be altered as needed. For example, in FIG. 29A, the spring 158 is not at the center of the button 156.

In FIGS. 26 and 27, a vent 162 is closed by depressing the manually actuated actuator or button 156. A seal stem 164 located on the underside of the plunger mount 160 will cover and substantially seal the vent 162 after the button 156 is depressed. The stem seal 164 may be located at the center of the underside of the plunger mount 160. To ensure a sufficient contact pressure between the vent 162 and the seal stem 164, a seal stem spring 166 mounted between the stem seal 162 and the plunger mount 160 pushes the seal stem 164 down onto the vent 162. In other embodiments, the seal stem may be made of a sufficiently compressible material such that a spring is not needed. One of ordinary skill in the relevant art will understand that by using a larger number of stem seals or by using a larger stem seal, multiple vents could be closed by the depression of a single button. Additionally, by modifying the length and position of the spring and stem seal, the system could be designed to close the vent before, during, or after the blisters are emptied.

In at least some embodiments of the invention, a stem seal 164 is used to close the waste sump vent 102 after the third button 22 is pressed. The stem seal 164 closes the waste sump vent 102 after a sufficient quantity of air has passed through the membrane 78 and into the waste sump 80 such that the membrane 78 has been adequately dried, but before the elution buffer has reached the membrane 78 and before the elution blister 44 is empty. By closing the waste sump vent 102, the stem seal 164 will redirect the flow of fluids coming through the membrane 78 into the reaction chamber pathway 100. As indicated above, a Laplace valve 101 may be located in fluid communication between the membrane 78 and the waste sump 80 which is larger and has a lower valve-opening pressure than a second Laplace valve connected to the reaction chamber pathway 100. Once the waste sump vent 102 is closed, however, no further fluids may enter the waste sump 80, and the pressure in the system will increase to the point where the valve to the reaction chamber pathway 100 opens. As the elution blister 44 continues to empty, the elution buffer will flow across the membrane 78; into the reaction chamber pathway 100; into the reconstitution chambers 48b, 48c; and into the reaction chambers 24b, 24c in a manner consistent with the disclosure above. In an alternative embodiment not shown in the drawings, the reaction chamber vents will be positioned such that a further stem seal can close the reaction chamber vents after the reaction chambers are full. In such an embodiment, the plungers 152 emptying the elution blister 44 and the negative control blister 46 continue to depress after the reaction chambers 24 are full. To enable this, Laplace valves could be employed with an activating pressure higher than the reaction chamber Laplace valves and connected to overflow chambers, which could be inflatable and without vents. Accordingly, all vents of the device would be sealed, and any potentially hazardous chemicals therein would be unable to escape.

In FIGS. 30A and 30B, a fluid collection device 168 is below and in fluid contact with the blister. The rupture pin 148 is located inside the fluid collection device 168. The fluid collection device 168 may be generally concave and bowl shaped. At the bottom of the fluid collection device 168 is an outlet port 170 where blister fluid can drain. Grooves 172 may be located on the bottom of the fluid collection device 168 to direct the fluid into the outlet port 170. The fluid collection device 168 may have a substantially flat rim 174 to adhere the blister to the collection device, creating a seal between the flat rim 174 and the blister. In some embodiments, the plunger 152 fits snugly into fluid collection device 168.

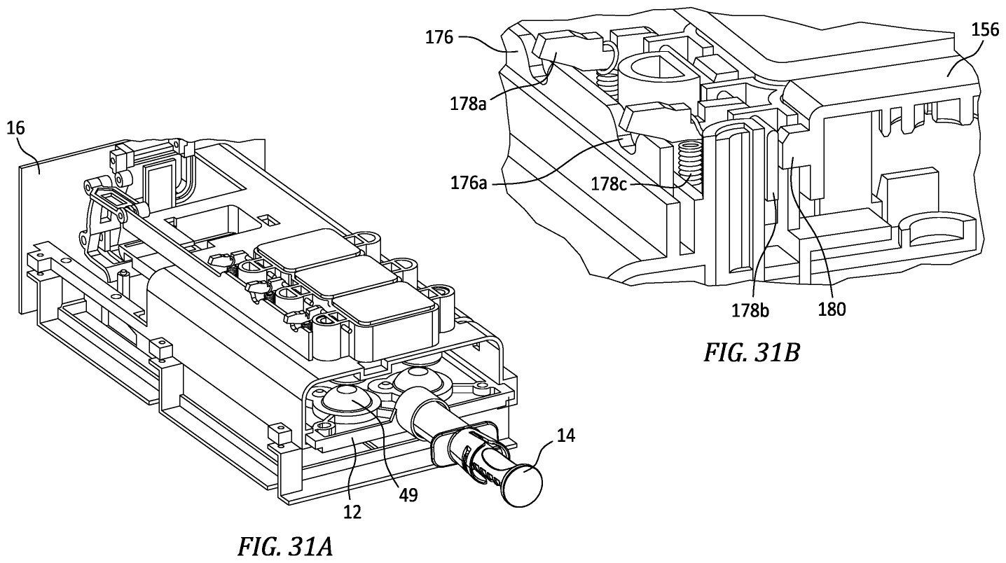

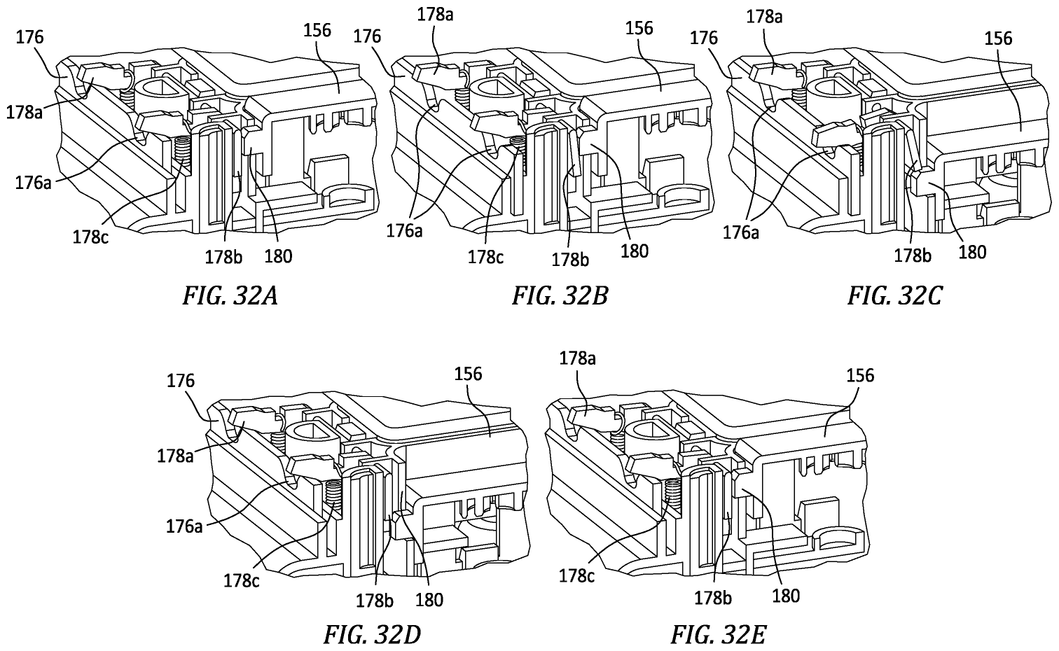

In FIG. 31-32, a locking mechanism which may be used with the device 10 is shown comprising a lock-release bar 176; a lock 178 comprising a lock arm 178a, lock tab 178b, and lock spring 178c; and a lock engagement tab 180 on button 156. In the illustrated embodiment, the locking device is part of the base station 16 into which the device is inserted. Initially, the lock release bar 176 prevents engagement of the locks preventing lock arm 178a from pivoting. In addition, lock engagement tab 180 on button 156 blocks lock tab 178a from pivoting. When the cartridge 12 is installed in the base station 16, the lock release bar 176 moves into a second position where a recess 176a in the lock release bar 176 allows the lock arm 178a to move, and the bar no longer blocks pivoting movement of lock arm 178a. The lock spring 178c urges the locking tab 178b into contact with the lock engagement tab 180 of the button 156 but allows relative movement thereof. In the first actuated position, the latch spring urges the locking tab into a locked position securing the lysis-wash actuator in the first actuated position. When button 156 is moves to the actuated position, lock engagement tab 180 of the button no longer blocks pivoting movement of lock tab 178b. Lock spring 178c urges the lock tab 178b into a locked position by pulling on lock arm 178a from below pivoting lock 178, with the distal end of lock arm 178a lowering and the distal end of lock tab 178b moving into a biased position above engagement tab 180 of button 156, securing the button 156 in the locked position. To unlock, the lock release bar 176 is moved, raising the distal end of the lock arm 178a and pivoting the lock tab 178b out of the path of the engagement tab 180. The button 124 used to release the latch 122 holding the cartridge 12 in the base station 16 may also move the lock release bar 176 into the unlock position.

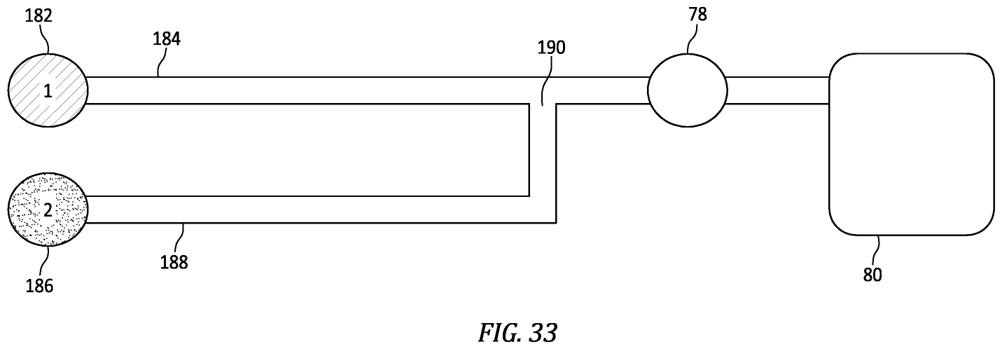

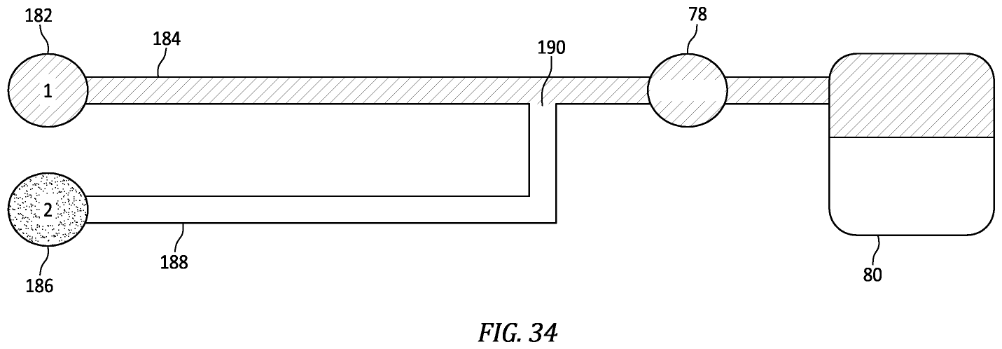

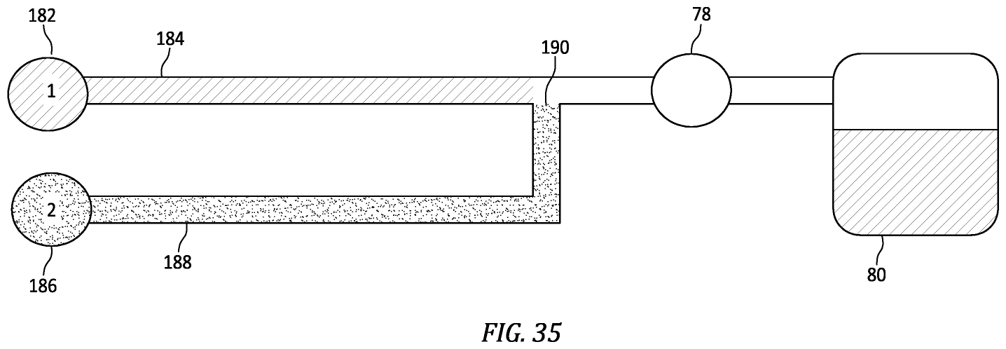

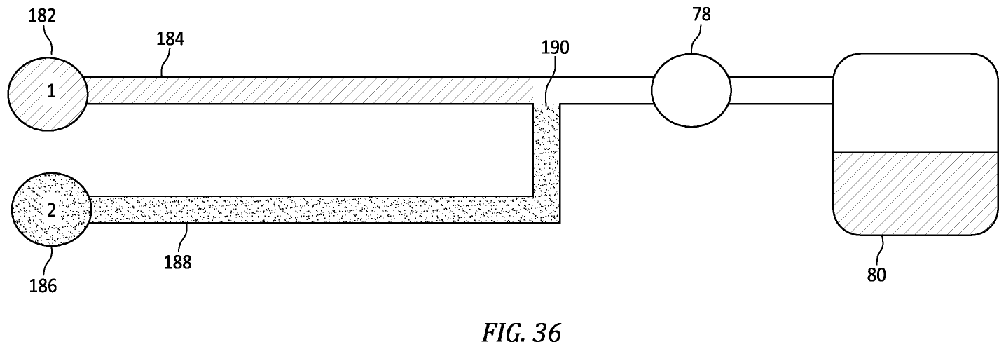

In FIGS. 33-36, the schematic for a dead-leg conduit is shown in four sequential stages of operation. The dead-leg conduit comprises a first blister 182, a first blister conduit 184, a second blister 186, a second blister conduit 188, a solid-state membrane 78, and a waste chamber 80. In the first stage, shown in FIG. 33, both blisters are filled with fluid as neither has been depressed. When the first blister 182 is depressed, as shown in FIG. 34, the fluid in the first blister 182 passes down first blister conduit 184 and through to the membrane 78 into the waste chamber 80. However, because the second blister conduit 188 is a dead-leg, i.e. without a vent, the fluid does not pass down the second blister conduit 188; the second blister conduit 188 is blocked by air, which in a narrow microfluidic channel, cannot be bypassed. Next, as shown in FIG. 35, the second blister 186 is depressed, and the fluid in the second blister 186 forces the air in the second blister channel 188 through the membrane 78 and into the waste chamber 80. In doing so, it pushes fluid from the first blister located at or downstream of the fluid conduit junction 190 to flow through the membrane 78 and into the waste chamber 80. However, the air and second blister fluid do not travel into the first fluid conduit 184, which does not have a vent and is blocked. Finally, as shown in FIG. 36, the button is depressed further and fluid from the second blister 186 flows downstream through junction 190, across membrane 78, and into waste chamber 80. However, due to the behavior of fluids in a microfluidic environment, the fluid from the second blister 186 does not mix to any significant degree with the fluid from the first blister 182 until reaching waste chamber 80.

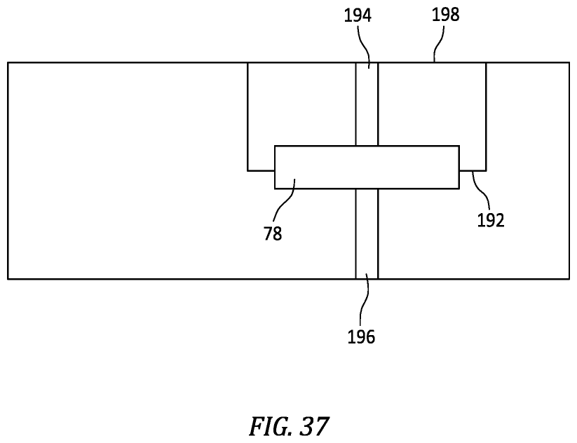

In FIG. 37, a configuration of the membrane 78 is shown. The membrane 78 is inside a recess or nest 192. A fluid inlet 194 and a fluid outlet 196 are in fluid contact with the membrane 78 on opposite sides thereof. A nest cap 198 helps to hold the membrane 78 in place and/or control the flow of liquid through the membrane.

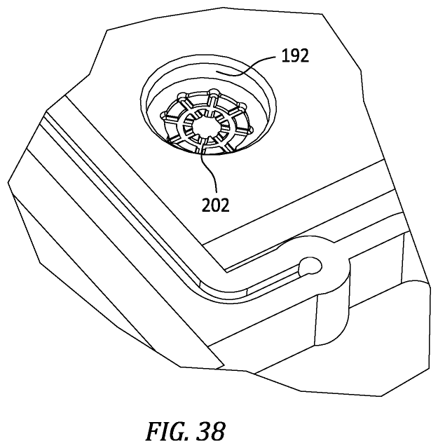

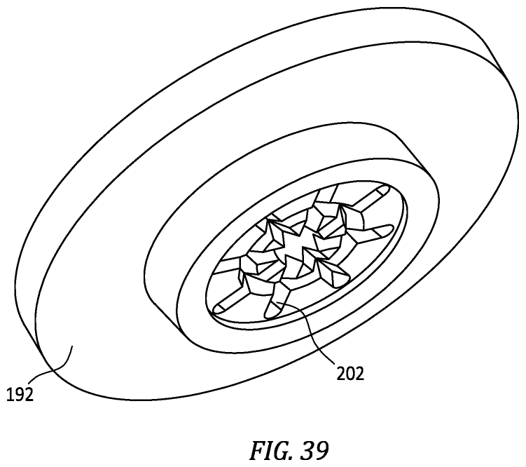

In FIGS. 38-39, examples of mechanisms to control the flow across the membrane 78 are shown. In order to increase the capture efficiency of the membrane, and in order to increase the flow rate across the membrane, the fluid passing over the membrane must pass as large a surface area as possible. In addition, the flow over the membrane must be substantially uniform across the membrane. It is advantageous to wet substantially the entire membrane because the membranes, once wet, allow fluids to pass through it more easily.

FIGS. 38 and 39 show the nest and nest cap configuration. The sides of the nest cap 198 and nest 192 which contact the membrane 78 have a pattern of grooves or spoked wetting lines 202. The fluid will travel down the wetting lines 202 before entering the membrane 78, substantially increasing the uniformity of flow across the membrane 78 and increasing the flow rate. The grooves are placed both radially and annularly to ensure substantially the entire membrane is wetted, preferably to use substantially every fiber of the membrane, or otherwise to maximize the capture of nucleic acids.

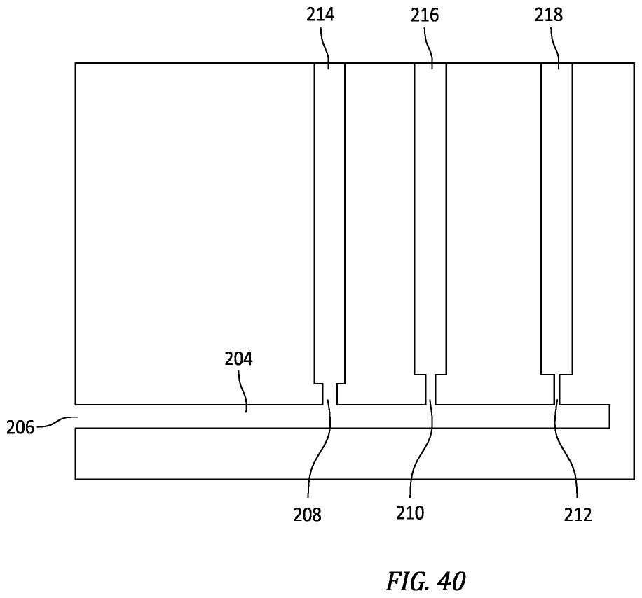

In FIG. 40, a schematic view of the Laplace valves with three holes of decreasing size that can be used in the cartridge of FIG. 1 to control progressive flow into the waste sump and then into the reaction chambers is illustrated. A Laplace valve is a fluidic device that utilizes a relatively small hole through a relatively thin membrane to prevent fluid from entering until the pressure differential through the device rises above a threshold or valve-opening pressure. Threshold pressure is dependent on hole size and physical properties of the fluid being transported. This phenomenon can be used in a microfluidic device where one channel feeds multiple downstream channels and control of the sequence of filling each downstream channel is desired. A Laplace valve may be positioned at each intersection of each downstream channel preventing fluid from entering that channel until the fluid pressure threshold is achieved. Sequencing can be controlled through logical schematic layout or by varying the size of the through holes thus changing the threshold pressure of each valve. As pressure rises fluid will enter the valves with larger holes before those with smaller holes. Sequencing can also be controlled by varying the through hole size. As pressure in the feeding channel increases, Laplace valves with larger holes will allow fluid to enter before those with smaller holes.

The Laplace valves shown in the schematic of FIG. 40, is a fluid system consisting of feeding channel 204 originating at the fluid inlet 206 and splitting at each of a first Laplace valve 208, a second Laplace valve 210, and a third Laplace valve 212. The diameter decreases from the first Laplace valve 208 to the second Laplace valve 210 and from the second Laplace valve 210 to the third Laplace valve 212. The first Laplace valve 208 is largest and the third Laplace valve 212 is smallest. When the system is filled with fluid from the fluid inlet, the first channel 214 fills first, then the second channel 216, then the third channel 218. Though the Laplace valves shown in FIG. 40 are linear, it will be understood by those of ordinary skill that the feeding channel or channels may be in any of numerous different shapes and/or configurations that are currently known or that later become known.

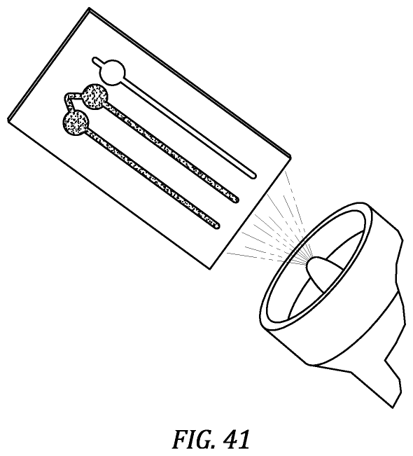

In FIG. 41, a sample device showing how the excitation light substantially does not exit the device through surfaces parallel to the excitation light direction, but will exit the device through surfaces normal to the direction of the excitation light. In addition, the two reaction chambers illuminated with green fluorescent light, as indicated by speckling, show a positive result and a positive control, whereas the darker reaction chamber has not been excited by the excitation light, as the negative control does not react.

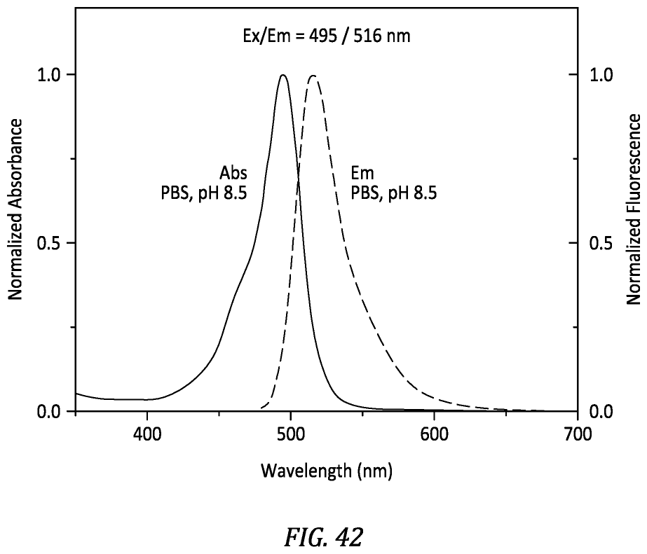

FIG. 42 shows excitation and fluorescence spectral responses, in solid and dashed lines respectively, for Calcein. Calcein is an indicator of LAMP reactions, the spectral output and fluorescence of a test chamber with Calcein is increased after a successful LAMP reaction. Because no LAMP reaction will occur if the target nucleic acid sequence is not present, this fluorescent reaction indicates a positive result, and the lack of fluorescence indicates a negative result. There should be a geometrical separation between the fluorescing and stimulating light, and it is also beneficial that there be a difference of the two wavelengths which is observable by the human eye. By using an illumination wavelength of about 470 nm and a fluorescing wavelength of about 510 nm, there is an observable color difference for the typical human observer: the human eye's color separation ability is maximal near these wavelengths.

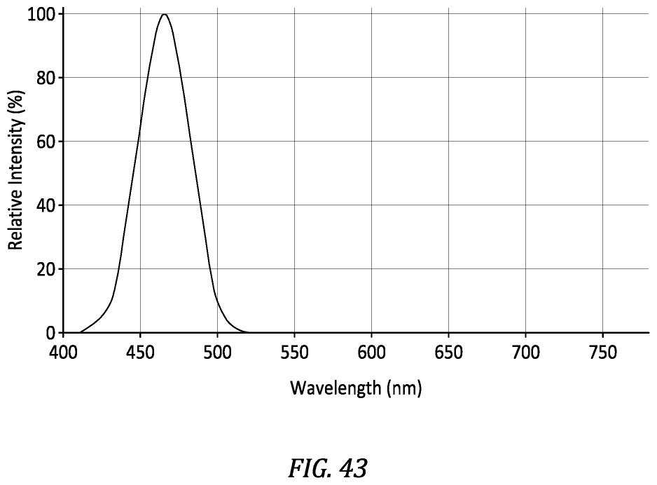

FIG. 43 shows the relative intensity of a stimulating LED from Wurth Elektronik (part number: 155124BS73200A). The LED spectral output is peaked at about 470 nm which is below the peak of the Calcein excitation sensitivity, but this allows for visual color discrimination by the human eye. Because samples with amplified DNA or RNA will absorb different amounts of light, the presence or absence of an amplified DNA or RNA sample will be identifiable when compared against a control positive and negative result. Other LEDs could be used. For example, if the light coming from the LED could be sufficiently shielded from the viewer, a stimulation wavelength of about 485 to about 495 nm would produce maximal fluorescence from Calcein. However, if the stimulating light were directed towards the observer, the color separation might not be observable and it would be difficult for the human eye to distinguish between a positive and negative result. One of ordinary skill would appreciate based on the teachings herein that, based on the ability of the cartridge to properly contain the stimulating light, the wavelength of the stimulating light may be altered to balance between creating an intense fluorescent response and ensuring the fluorescent color may be distinguished from any stimulating light which leaks out.

While Calcein and such stimulating lights are a method of detecting an amplification reaction, one of ordinary skill would appreciate based on the teachings herein that Calcein is only one such indicating chemical, and any other indicators which are known or may become known could be used, in conjunction with techniques which are known or which may become known to identify the results of amplification reactions. It is generally preferred that a colored dye be used to ensure simplicity of the test, in that an untrained observer will understand the change in color, but other methods of detecting amplification reactions may also be used.

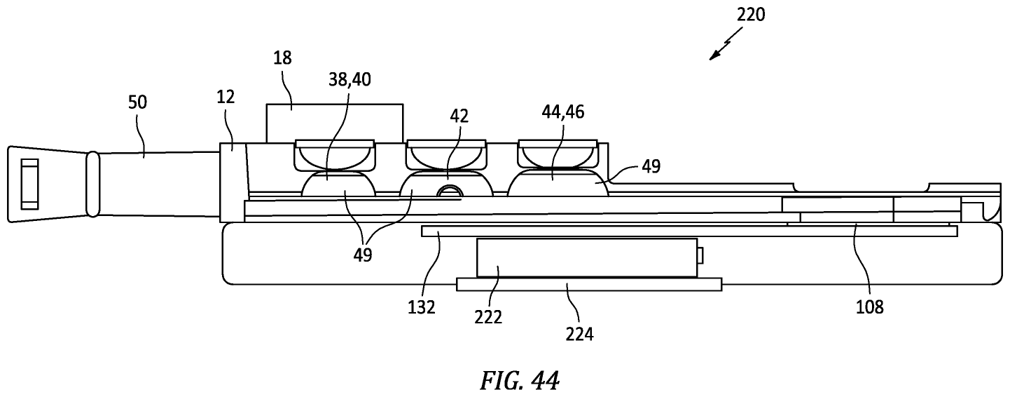

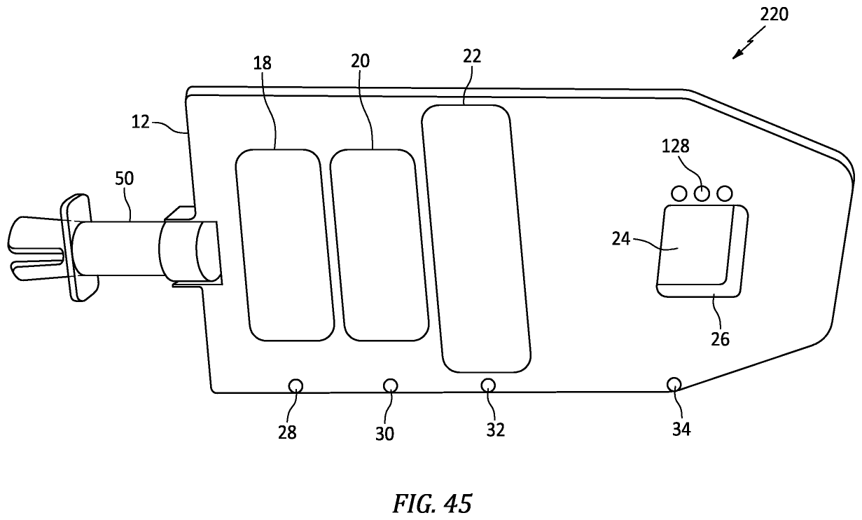

FIGS. 44 and 45 show another disposable cartridge indicated generally by the numeral 220. The disposable cartridge 220 comprises a device body 12; a first button indicator 28 indicating when to push a first button 18 thereby compressing the lysis blister 38 and the first wash blister 40, a second button indicator 30 indicating when to press a second button 20 configured to compress the second wash blister 42, and a third button indicator 32 indicating when to press a third button 22 thereby compressing the negative control blister 46 and elution blister 44; a results window 26, a reaction complete indicator 34, a main printed wiring board 132, a heating element 108, and a power source 222 such as a battery. In embodiments where the power source is a battery, the device 220 may include a battery cover 224 and a plastic tab may prevent electricity from flowing from the batteries through to the device until it is removed, preventing the batteries from draining sooner than intended. The operation of the device is as described above, except that the device body does not need to be inserted into a base station, all the relevant components of the base station are included in the disposable device.

As may be recognized by those of ordinary skill in the pertinent art based on the teachings herein, numerous changes, improvements, modifications, additions and deletions may be made to the above-described and other embodiments of the present invention without departing from the scope of the invention. For example, the components of the device may take any of numerous different configurations and may be made of any of numerous materials that are currently known or later become known, and features may be added to or removed therefrom, without departing the from the scope of the invention. Accordingly, this detailed description of embodiments is to be taken in an illustrative as opposed to a limiting sense.

What is claimed is:

1. A device for detecting nucleic acids in a biological sample, comprising:
a sample port for receiving therein a biological sample;
a solid-state membrane configured to capture nucleic acids in the biological sample passed across the membrane;
a sample conduit in fluid communication between the sample port and the solid-state membrane;
a lysis station in fluid communication with the sample conduit and including a lysis agent therein;
a wash station in fluid communication with at least one of the sample conduit or the solid-state membrane and including a wash solution therein;
an elution station in fluid communication with at least one of the sample conduit or the solid-state membrane and including an eluent therein;
a waste chamber located downstream of the solid-state membrane; and
one or more reaction chambers located downstream of the solid-state membrane;
wherein the sample port, lysis station and sample conduit are configured to mix the sample and lysis agent to form a sample-lysis mixture, pass the sample-lysis mixture across the solid-state membrane to capture nucleic acids in the biological sample therein, and receive the remainder of the sample-lysis mixture in the waste chamber, the wash station is configured to introduce the wash solution into at least one of the sample conduit or solid-state membrane following the sample-lysis mixture to purify nucleic acids captured on the solid-state membrane, the wash solution from the solid-state membrane is received in the waste chamber, and the elution station is configured to pass the eluent across the solid-state membrane, elute captured nucleic acids from the solid-state membrane, and pass the captured nucleic acids into one or more reaction chambers configured for amplifying and detecting the captured nucleic acids therein.

2. A device as defined in claim 1, further comprising a lysis leg extending in fluid communication between the lysis station and the sample conduit at a sample-lysis junction and configured to direct a flow of the lysis agent from the lysis station into the sample conduit and allow the lysis agent to mix with the sample and form the sample-lysis mixture, and a wash leg extending in fluid communication between the wash station and at least one of the sample conduit or the solid-state membrane at a sample-wash junction and configured to direct a flow of the wash solution from the wash station behind the sample-lysis mixture.

3. A device as defined in claim 2, further comprising a static mixer in fluid communication between the sample-lysis junction and the solid-state membrane and configured to mix the sample and lysis agent prior to passage across the solid-state membrane.

4. A device as defined in claim 1, further comprising a base station for receiving the device, wherein the base station includes a ramp, a sled mounted on the ramp, and a heater mounted on the sled, wherein upon receiving the device into the base station, the sled is connectable to the device and movable therewith between a non-operational position and an operational position, and during movement between the non-operational position and the operational position, the heater is moved from a non-operational position out of contact with the device to an operational position in contact with the device and adjacent to the reaction chamber for incubating the captured nucleic acids within the reaction chamber.

5. A device as defined in claim 1, wherein the elution station includes a first sealed eluent chamber containing a first eluent, a first elution leg in fluid communication between the first elution station and at least one of the sample conduit or the solid-state membrane, a second sealed eluent chamber containing a second eluent, a second elution leg in fluid communication between the second elution station and at least one of the sample conduit or the solid-state membrane, and an elution actuator movable between a non-actuated position and an actuated position, wherein in the actuated position the first and second eluents are released from the first and second eluent chambers and into the first and second elution legs.

6. A device as defined in claim 5, wherein the elution actuator substantially simultaneously releases the first and second eluents from the first and second eluent chambers, the second elution leg is longer than the first elution leg to thereby allow the first eluent to pass across the solid-state membrane prior to passage of the second eluent across the solid-state membrane.

7. A device as defined in claim 5, wherein the wash solution and/or lysis agent leaves an evaporative contaminant on the solid-state membrane after passage therethrough, and the first elution leg contains a volume of air therein such that upon releasing the first eluent into the first elution leg, the volume of air in the first elution leg is passed across the solid-state membrane and is sufficient to substantially evaporate the evaporative contaminant and thereby prevent contamination of the first eluent and captured nucleic acids received within the reaction chamber.

8. A device as defined in claim 7, wherein the evaporative contaminant is ethanol, and the volume of air in the first elution leg is sufficient to substantially evaporate the ethanol in and about the solid-state membrane prior to passage of the first eluent across the membrane to substantially prevent ethanol poisoning of the reaction chamber.

9. A device as defined in claim 5, wherein the elution actuator includes a manually-engageable portion, a plunger mount, a first elution plunger engageable with the first eluent chamber, a second elution plunger engageable with the second eluent chamber, a plunger spring defining a plunger spring force and mounted between the manually-engageable portion and the plunger mount, and a latch, wherein upon manually moving the manually-engageable portion from a non-actuated position to a first actuated position, the latch secures the manually-engageable portion in the first actuated position where the first elution plunger partially dispenses the first eluent chamber and the second elution plunger partially dispenses the second eluent chamber, and the plunger spring drives the plunger mount from the first actuated position to a second actuated position under the plunger spring force to further dispense the first and second eluents from the first and second eluent chambers, respectively.

10. A device as defined in claim 9, further comprising a waste chamber vent in fluid communication between the waste chamber and ambient atmosphere; a waste vent seal movable between an open position allowing fluid to flow out of the waste chamber vent and thereby allow fluid to flow into the waste chamber, and a closed position sealing the vent and thereby preventing fluid from flowing into the waste chamber; and a waste vent seal spring urging the waste vent seal in a direction from the open position to the closed position.

11. A device as defined in claim 10, wherein the waste vent seal is mounted on the plunger mount, and the waste vent seal spring is mounted between the waste vent seal and the plunger mount, and upon movement of the manually-engageable portion into the first actuated position, the plunger spring and the waste vent seal spring urge the waste vent seal into the closed position to thereby seal the waste chamber vent.

12. A device as defined in claim 1, further comprising a reaction chamber valve in fluid communication between the solid-state membrane and the reaction chamber, wherein the reaction chamber valve is (i) closed to prevent fluid flow into the reaction chamber when a fluid pressure between the solid-state membrane and the reaction chamber valve is below a valve-opening pressure and (ii) is open to allow fluid flow into the reaction chamber when the fluid pressure between the solid-state membrane and the reaction chamber valve is above the valve-opening pressure.

13. A device as defined in claim 12, wherein the reaction chamber valve is a Laplace or burst valve.

14. A device as defined in claim 12, further comprising a waste chamber vent in fluid communication between the waste chamber and ambient atmosphere; and a waste vent seal movable between an open position allowing fluid to flow out of the waste chamber vent and thereby allow fluid to flow into the waste chamber, and a closed position sealing the vent and thereby preventing fluid from flowing into the waste chamber; wherein movement of the waste chamber vent seal into the closed position causes the fluid pressure between the solid-state membrane and reaction chamber valve to exceed the valve-opening pressure of the reaction chamber valve and thereby allow fluid flow from the solid-state membrane into the reaction chamber.

15. A device as defined in claim 12, further comprising a first reaction chamber, a second reaction chamber, a first reaction chamber vent in fluid communication between the first reaction chamber and ambient atmosphere and configured to allow gas but substantially prevent liquid flow therethrough, and a second reaction chamber vent in fluid communication between the second reaction chamber and ambient atmosphere and configured to allow gas but substantially prevent liquid flow therethrough.

16. A device as defined in claim 15, wherein each of the first and second reaction chamber vents includes a hydrophobic vent membrane that allows gas but substantially prevents liquid flow therethrough.

17. A device as defined in claim 1, further comprising a plurality of actuators, wherein each actuator is manually movable from a non-actuated position to an actuated position, each of the lysis station, wash station and elution station includes a sealed chamber including a frangible or breakable wall and containing therein the lysis agent, wash solution or eluent, respectively, and wherein upon movement of each actuator from the non-actuated to the actuated position, one or more of the frangible or breakable walls is broken to release at least one of the lysis agent, wash solution and/or eluent from its respective sealed chamber.

18. A device as defined in claim 1, wherein the solid-state membrane includes an inlet side and an outlet side, and the device further comprises a membrane inlet located on the inlet side of the solid-state membrane and in fluid communication between the solid-state membrane and at least one of the sample conduit or elution station, and a membrane outlet located on the outlet side of the solid-state membrane in fluid communication between the solid-state membrane and at least one of the waste chamber or reaction chamber, wherein the membrane inlet defines a plurality of inlet fluid channels configured to facilitate a flow of fluid across the inlet side of the solid-state membrane, and the membrane outlet includes a plurality of fluid outlet channels therein configured to facilitate a flow of fluid across the outlet side of the solid-state membrane.

19. A device as defined in claim 1, further comprising a body including the at least one reaction chamber therein, wherein at least a portion of the body including the reaction chamber is substantially transparent, and includes a substantially transparent top surface extending over the reaction chamber and two substantially transparent side surfaces extending downwardly from the top surface along opposite sides of the reaction chamber relative to each other, wherein the reaction chamber is visually observable in a viewing direction through the substantially transparent top surface, and further comprising a stimulating light source located adjacent to a substantially transparent side surface and configured to transmit stimulating light through the side surface and reaction chamber in a direction substantially lateral to the viewing direction.

20. A device as defined in claim 19, wherein the index of refraction of the substantially transparent body and the index of refraction of the fluid in the reaction chamber are configured to facilitate the passage of the stimulating light from the body into the reaction chamber to generate fluorescing light in the reaction chamber such that the fluorescing light is emitted in substantially all directions and is observable in the viewing direction through the top surface of the body.

21. A device as defined in claim 20, wherein there is an observable difference to the human eye between the stimulating light and the fluorescing light to facilitate the ability of an observer to view the fluorescing light and distinguish it from any observed stimulating light.

22. A device as defined in claim 21, wherein the stimulating light defines a first wavelength within the range of about 425 nm to about 550 nm, and the fluorescing light defines a second wavelength greater than the first wavelength.

23. A device as defined in claim 1, further comprising a saliva collection swab for collecting saliva thereon and receivable within the sample port for introducing the saliva directly into the sample port and sample conduct for mixture therein with the lysis agent.

24. A device as defined in claim 23, wherein the saliva collection swab includes a plunger depressible against the saliva collection swab within the sample port to release saliva from the collection swab into the sample port and sample conduit, at least one of the saliva collection swab or the sample port includes a locking tab, and the other of the saliva collection swab or sample port includes a corresponding locking recess or aperture configured to receive the locking tab and retain the swab within the sample port with the plunger depressed against the swab to facilitate release of saliva therefrom and into the sample port.

25. A device as defined in claim 1, further comprising a reaction chamber vent in fluid communication between the reaction chamber and ambient atmosphere, wherein the reaction chamber vent defines a venting length extending between the reaction chamber and ambient atmosphere and a venting cross-sectional area, and the venting cross-sectional area is sufficiently small compared to the venting length to create a saturation gradient between the reaction chamber and ambient atmosphere to slow the evaporation of liquid from and prevent the entry of atmospheric air into the reaction chamber.

26. A device as defined in claim 1, further comprising a body defining therein the sample conduit, a lysis leg extending in fluid communication between the lysis station and the sample conduit and configured to direct a flow of the lysis agent from the lysis station into the sample conduit, a wash leg extending in fluid communication between the wash station and the sample conduit upstream of the lysis leg and configured to direct a flow of the wash solution from the wash station into the sample conduit behind the sample-lysis mixture, an elution leg extending in fluid communication between the elution station and the solid-state membrane, a viewing window overlying the reaction chamber and allowing visual observation of the reaction chamber therethrough in a viewing direction, a heater mounted to the body adjacent to the reaction chamber and configured to heat the reaction chamber, a stimulating light source configured to transmit stimulating light into the reaction chamber in a direction lateral to the viewing direction, and a power source connected to the heater and light source and configured to provide power thereto.

27. A device as defined in claim 1, further comprising a plurality of reaction chambers, and reagents located within or in fluid communication with the reaction chambers and configured to mix with eluted captured nucleic acids flowing from the solid-state membrane and into the reaction chambers and amplify the captured nucleic acids therein.

28. A device as defined in claim 1, further comprising a first reaction chamber, a second reaction chamber, a first reaction chamber valve in fluid communication between the solid-state membrane and the first reaction chamber and defining a first valve opening pressure, a second reaction chamber valve in fluid communication between the solid-state membrane and the second reaction chamber and defining a second valve opening pressure greater than the first valve opening pressure, wherein upon substantially filling the first reaction chamber, the fluid pressure at the second reaction chamber valve exceeds the second valve opening pressure to open the second reaction chamber valve and fill the second reaction chamber.

29. A device as defined in claim 28, further comprising a waste chamber vent in fluid communication between the waste chamber and ambient atmosphere; and a waste vent seal movable between an open position allowing fluid to flow out of the waste chamber vent and thereby allow fluid to flow into the waste chamber, and a closed position sealing the vent and thereby preventing fluid from flowing into the waste chamber; wherein in the open position of the waste vent seal, the fluid pressure at the first and second reaction chamber valves is below their valve opening pressures to prevent fluid flow into the first and second reaction chambers and direct fluid flow into the waste chamber, and in the closed position of the waste vent seal, the fluid pressure at the first and second reaction chamber valves is greater than the first valve opening pressure to allow filling of the first reaction chamber, and upon substantially filling the first reaction chamber, the fluid pressure at the second reaction chamber valve is greater than the second valve opening pressure to allow filling of the second reaction chamber.

30. A device for detecting nucleic acids in a biological sample, comprising:
first means for receiving therein a biological sample;
second means for capturing nucleic acids in the biological sample;
third means in fluid communication between the first means and the second means for directing the biological sample to the second means;
fourth means in fluid communication with the third means for introducing a lysing agent therein with the biological sample and passing a sample-lysis mixture across the second means to capture nucleic acids in the biological sample therein;
fifth means in fluid communication with at least one of the second means or the third means for introducing a wash solution therein following the sample-lysis mixture and passing the wash solution across the second means to purify nucleic acids captured therein;
sixth means in fluid communication with at least one of the second means or the third means for introducing an eluent across the second means and eluting captured nucleic acids from the second means;
seventh means located downstream of the second means for receiving the remainder of the sample-lysis mixture that passes through the second means and the wash solution that passes through the second means; and
at least one eighth means located downstream of the second means for receiving the captured nucleic acids from the second means and amplifying and detecting the captured nucleic acids therein.

31. A method for detecting nucleic acids in a biological sample, comprising:
receiving a biological sample through a sample port and into a sample conduit in fluid communication between the sample port and a solid-state membrane for capturing nucleic acids in the biological sample and amplifying and detecting the captured nucleic acids therein in at least one reaction chamber;
introducing a lysing agent into the sample conduit, mixing the lysing agent with the sample to form a sample-lysis mixture, passing the sample-lysis mixture across the solid-state membrane and capturing nucleic acids in the biological sample therein, preventing the flow of the sample-lysis mixture that passes across the solid-state membrane into the at least one reaction chamber, and receiving the remainder of the sample-lysis mixture that passes across the solid-state membrane in a waste chamber;
introducing a wash solution into at least one of the sample conduit or solid-state membrane following the sample-lysis mixture, passing the wash solution across the solid-state membrane and purifying nucleic acids captured from the sample-lysis mixture therein, preventing the flow of the wash solution into the reaction chamber, and receiving the wash solution that passes through the solid-state membrane in the waste chamber; and
introducing an eluent across the solid-state membrane and eluting captured nucleic acids from the solid-state membrane, substantially preventing the captured nucleic acids from flowing into the waste chamber, directing the captured nucleic acids into the at least one reaction chamber, and amplifying and detecting the captured nucleic acids in the at least one reaction chamber.
